(19) United States
(12) Patent Application Publication
(10) Pub. No.: US 2007/0151563 A1
(43) Pub. Date: Jul. 5, 2007

(54) Apparatus and method for controlling gas-delivery mechanism for use in respiratory ventilators

(76) Inventors: Kenji Ozaki, Kobe-shi (JP); Seiichi Shin, Moriya-shi (JP); Kazutoshi Soga, Kobe-shi (JP)

Correspondence Address: HARNESS, DICKEY & PIERCE, P.L.C., P.O. BOX 8910, RESTON, VA, 20195, US

(21) Appl. No.: 11/315,337

(22) Filed: Dec. 23, 2005

(51) Int. Cl. A61M 16/00; A62B 7/00

(52) U.S. Cl. 128/204.23; 128/204.21

(57) ABSTRACT

A method of controlling a mechanical ventilator is provided which is capable of increasing a stability margin in the controlling of a gas-delivery mechanism for the mechanical ventilator. The flow rate F of an assisting gas is measured, and an observer 54 estimates a flow rate \hat{F} of the assisting gas. A difference ΔF between the measured flow rate F and estimated flow rate \hat{F} is then determined, and information on a patient's respiratory effort pressure Pₘᵤₛ is obtained. A target pressure Pᵢₙ for controlling a gas-delivery mechanism 20 is calculated on the basis of this information. When the target pressure Pᵢₙ is calculated on the basis of the flow-rate difference ΔF, an allowance with respect to the stability limit of the overall system 14 can be increased. This enables the runaway to rarely occur even when an actual overall system is varied. Moreover, the responsibility of assist respiration can be improved as compared with that of the related art PAV method.

### BACKGROUND OF THE INVENTION

1. Field of the Invention

The present invention relates to a method and apparatus for controlling a gas-delivery mechanism for use in mechanical ventilators applied to a patient with spontaneous breathing effort.

2. Description of the Related Art

There has been employed a Proportional Assist Ventilation method (hereinafter called PAV) as a ventilation mode or control method for mechanical ventilators. The method is related to how to supply air or oxygen-enriched air (hereinafter called “gas”) systematically during inspiratory period of patients with spontaneous breathing, and more specifically to how to control gas supply mechanism of mechanical ventilators (hereinafter simply called “gas-delivery mechanism”).

An overall system 5 including a patient's respiratory system 2 and a mechanical ventilator 1 is illustrated in FIG. 26, which is a block diagram illustrated to conceptually clarify the PAV method. The mechanical ventilator 1 realizing PAV generally comprises a gas-delivery mechanism 3 and a control calculation part 4 to give a command for the gas-delivery mechanism 3. The control part 4 calculates and determines a target pressure of the gas-delivery mechanism 3 using measured patient's flow-rate.

The control part 4 comprises three calculation elements. A first element of the three calculates a flow-rate-assist amount that is a patient's flow-rate multiplied with a predetermined so-called flow-rate-assist gain K_{fa}. A second element of the three calculates a volume-assist amount that is obtained by multiplying a resulted patient's inspired volume V with a predetermined so-called Volume-assist gain K_{Va}, which corresponds to (K_{Va}×F)/s, where s represents Laplace Transform operator. A third calculation element of the three adds the two signals of the flow-rate-assist amount and the volume-assist amount and gets a target pressure signal denoted as Pᵢₙ. In general, flow-rate-assist gain K_{fa} is \hat{R}×α and volume-assist gain K_{Va} is Ê×α where \hat{R} and Ê are estimated or identified respiratory resistance and elastance, respectively, and α is a so-called assist ratio in the PAV method. Hereinafter when \hat{R}=R and Ê=E, an assist ratio α is denoted as A, wherein R and E represent real patient's respiratory resistance and elastance, respectively.

The target pressure signal Pᵢₙ calculated in the control part is transmitted to the gas-delivery mechanism as its command signal. The gas-delivery mechanism 3 generates a patient-assist pressure Pᵥₑₙₜ based on the command signal Pᵢₙ. As described in Japanese Examined Patent Publication JP-B2 2714288, for example, the gas-delivery mechanism 3 can generate an assisting gas pressure Pᵥₑₙₜ corresponding to a linear amplified pressure Pₘᵤₛ that represents patient's respiratory effort pressure when the flow-rate-assist gain K_{fa} and volume-assist gain K_{Va} are appropriately set, which corresponds an ideal case with \hat{R}=R, Ê=E, K_{fa}=\hat{R}×α, K_{Va}=Ê×α,

The FIG. 27 shows a graphical relation between the spontaneously inspired gas volume Vₘᵤₛ and ventilator assisting gas volume Vₐₛₜ with the overall system 5 of the existing PAV method. As mentioned above, the ventilator-assisting gas pressure Pᵥₑₙₜ is amplified Pₘᵤₛ, which is a resultant pressure of patient spontaneous breathing effort during time course of patient's inspiratory period. Ideally if we can put \hat{R}=R, Ê=E, the amplification ratio will be 1/(1−A) where A is the assist ratio of α in this ideal case.

In the existing PAV method, flow-rate-assist gain K_{fa} and volume-assist gain K_{Va} should be set after getting precisely estimated patient's respiratory resistance \hat{R} and respiratory elastance Ê, respectively. For this purpose, the systematic identification method for \hat{R} and Ê has been shown as a related art in the Japanese Unexamined Patent Publication No. 11-502755 (1999).

As illustrated in the overall system 5 in FIG. 26, supplying pressure to patient's respiratory system corresponds to Pᵥₑₙₜ+Pₘᵤₛ and it is achieved so-called positive feedback configuration in the existing PAV technology. This positive feedback configuration causes a possibility to be unstable in an overall system of patients and mechanical ventilators. This unstable phenomenon is called runaway, which presents harmful influences with an extreme over assist.

Runaway will likely occur, when a flow-rate-assist gain or volume-assist gain is inadequately set, for example, an extremely unbalance value is set as compared with a real respiratory resistance and elastance, when the response characteristics of gas-delivery mechanism 3 is not sufficiently quick, when patient's conditions change, or another phenomena such as an effect of a disturbance occurs. If this phenomenon will happen, assist flow-rate will be amplified without any convergence value and should be divergent. This phenomenon causes some injures of patient's lung and/or respiratory airway and as a result it is obliged to stop ventilatory support during assist with the existing PAV technology.

The reason for easy occurrence of runaway is that the existing PAV overall system 5 has not enough stability margins, which causes to an unstable transient response. If the stability margin is not enough, the system is easily beyond a stable limit even though the system will have a slight change in its dynamic characteristics, and consequently runaway will be likely to happen. To prevent this phenomenon, it is necessary to set adequate values of flow-rate-assist gain K_{fa} and volume-assist gain K_{Va}, with the result that such selectable regions of these values are not so enough. This means it is not so easy to have good setting values.

### SUMMARY OF THE INVENTION

An object of the invention is to provide a method and apparatus for controlling a gas-delivery mechanism for use in a mechanical ventilator capable of having a large stability margin in the overall system including patients and the ventilator.

The invention provides a method for controlling a gas-delivery mechanism of a mechanical ventilator which supplies a gas containing oxygen having an assisting gas pressure Pᵥₑₙₜ corresponding to patient's respiratory effort pressure Pₘᵤₛ, the method comprising:

a flow-rate measuring step of measuring a flow rate F of an assisting gas supplied to a patient's respiratory airway;

a flow-rate estimation step of estimating a flow rate \hat{F} of an assisting gas to be supplied to the patient's respiratory airway when the assisting gas having an assisting gas pressure Pᵥₑₙₜ is supplied to the patient's respiratory airway, with the aid of flow-rate estimation means in which a patient's respiratory system is modeled;

a difference calculation step of calculating a flow-rate difference ΔF between the measured flow rate F and the estimated flow rate \hat{F}; and

a control value calculation step of calculating the target pressure Pᵢₙ based on the flow-rate difference ΔF and providing a signal representing the target pressure Pᵢₙ to the gas-delivery mechanism.

The assisting gas pressure Pᵥₑₙₜ is realized with the target pressure Pᵢₙ calculated based on Pₘᵤₛ which is un-measurable pressure. The invention comprises a flow-rate measuring step of measuring a patient's inspiratory gas flow F; a flow-rate-estimation step with a programmable model for patient's respiratory system in which the flow rate induced by the assisting gas pressure Pᵥₑₙₜ against the patient's respiratory system will be calculated as \hat{F}; the difference ΔF calculation step of calculating a difference between the measured F and the estimated \hat{F}; calculation step of calculating the target pressure value Pᵢₙ as the result of calculated flow-rate difference ΔF; and a control value calculation step of determining a target value Pᵢₙ of the gas-delivery mechanism.

According to the invention, the actual patient's flow rate F is measured and also the assisting gas flow-rate-estimation value \hat{F} is calculated at the estimation calculation step. The measured flow rate F will be affected by patient's respiratory effort pressure Pₘᵤₛ, but the estimated flow rate \hat{F} is not affected thereby. As a consequence, information of un-measurable patient's respiratory effort pressure Pₘᵤₛ can be obtained by calculating the flow-rate difference ΔF between the measured flow rate F and the estimated flow rate \hat{F}.

At the control value calculation step, the target pressure Pᵢₙ of the gas-delivery mechanism is calculated based on the flow-rate difference ΔF, and as a result, the target pressure Pᵢₙ also corresponds to the patient's respiratory effort pressure Pₘᵤₛ. Using the target pressure as mentioned above, the assisting gas can be supplied to the patient with the assisting gas pressure Pᵥₑₙₜ corresponding to the patient's respiratory effort pressure Pₘᵤₛ which will change instantaneously and successively.

In addition, as compared with the related-art PAV method in which the target pressure Pᵢₙ is determined based on only the measured assisting gas flow-rate signal F, in the invention, the overall system can prevent from being likely a positive-feedback configuration when the target pressure Pᵢₙ be calculated based on the flow-rate difference ΔF. The result can give a greater stability margin as compared with the related-art technology. As a consequence, runaway phenomena can be prevented from being introduced even in a case where a disturbance occurs, a gas-delivery mechanism has a fairly big delay, a user cannot set an appropriate flow-rate-assist or volume-assist gain, and/or a patient's respiratory condition will change successively. This can reduce the patient's excessive load caused by the runaway phenomena.

According to the invention, as mentioned above, the respiratory effort pressure Pₘᵤₛ is calculated based on the flow-rate difference ΔF. As a result, an objective of the proportional assist ventilation method to deliver an assisting gas having an assisting gas pressure Pᵥₑₙₜ proportional to the patient's respiratory effort pressure Pₘᵤₛ to the patient's respiratory airway is realized reliably.

In addition, the stability margin of the overall system can be bigger than the related-art PAV technology in which the measured assisting gas flow rate is directly multiplied by an assist gain, and thereby such a configuration can be realized that runaway phenomena cannot be likely to be occurred. As a consequence, runaway phenomena can be prevented from occurring even in the case where the actual overall system is changed because of disturbance effect, fairly big delay of the gas-delivery mechanism and/or successive changes of parameters in a patient's respiratory system. Accordingly a control method for a gas-delivery mechanism to reduce the patient's excessive load caused by runaway phenomena can be realized.

In the invention, it is preferable that the assisting gas flow rate \hat{F} to a patient is estimated based on a series of time-courses that the target pressure signal Pᵢₙ is calculated and thereafter transmitted to the gas-delivery mechanism, and then this gas-delivery mechanism consequently delivers the assisting gas having an assisting gas pressure Pᵥₑₙₜ to the patient.

According to the invention, the assisting gas flow rate \hat{F} is calculated so as to correspond to the time-response characteristics of the gas-delivery mechanism. In the flow-estimating part, for example, \hat{F} is calculated taking account of the transfer function with time-delay component, and as a result \hat{F} can be calculated more precisely. Moreover, with the feature, the invention can reduce a time difference and/or time lag between a patient's inspiratory duration and/or end-time and assisting gas delivering duration and/or ending time of gas delivery by the gas-delivery mechanism. This time difference and/or time lag between patient and ventilator is called as “an asynchrony” and the invention consequently prevent this phenomenon. With the feature, the invention furthermore reduce additional patient's breathing load.

The invention provides a control apparatus for controlling a gas-delivery mechanism of a mechanical ventilator which supplies a gas containing oxygen having an assisting gas pressure Pᵥₑₙₜ corresponding to patient's respiratory effort pressure Pₘᵤₛ, the method comprising:

flow-rate measuring means for measuring a flow rate F of an assisting gas supplied to a patient's respiratory airway;

flow-rate estimation means for estimating a flow rate \hat{F} of an assisting gas to be supplied to the patient's respiratory airway when the assisting gas having an assisting gas pressure Pᵥₑₙₜ is supplied to the patient's respiratory airway, in the flow-rate estimation means a patient's respiratory system being modeled;

difference calculation means for calculating a flow-rate difference ΔF between the measured flow rate F and the estimated flow rate \hat{F}; and

control value calculation means for calculating the target pressure Pᵢₙ based on the flow-rate difference ΔF and providing a signal representing the target pressure Pᵢₙ to the gas-delivery mechanism.

According to the invention, the control apparatus comprises flow-rate measuring means for patient's inspiratory gas flow F; flow-rate-estimation means with a programmable model for a patient's respiratory system in which the flow-rate resulting with the assisting gas pressure Pᵥₑₙₜ against the patient's respiratory system will be calculated as \hat{F}; flow-rate difference ΔF calculation means between measured flow rate F and estimated flow rate \hat{F}; control value calculation means of the target pressure signal Pᵢₙ as a result of calculated flow-rate difference ΔF and of determining the target pressure signal Pᵢₙ as a target-value signal for the gas-delivery mechanism.

Accordingly the invention provides a control apparatus for a gas-delivery mechanism of a mechanical ventilator supplying a gas containing oxygen. The control apparatus can realize assisting gas pressure Pᵥₑₙₜ corresponding to patient's respiratory effort pressure Pₘᵤₛ. This Pᵥₑₙₜ is realized with the target pressure Pᵢₙ calculated based on Pₘᵤₛ, which is un-measurable pressure. As mentioned above, the actual patient's flow rate F is measured and also the assisting gas flow rate estimation signal \hat{F} is calculated by the flow-rate estimation means. The measured flow-rate signal F will be affected by patient's respiratory effort pressure Pₘᵤₛ but the estimated flow rate \hat{F} is not affected thereby. As a consequence, a signal corresponding to un-measurable patient's respiratory effort pressure Pₘᵤₛ can be obtained by calculating the difference ΔF between the measured flow rate F and the estimated flow rate \hat{F}.

In the control value calculation means, the target pressure signal Pᵢₙ of the gas-delivery mechanism is determined based on the flow-rate difference ΔF, and as a result, the target pressure signal Pᵢₙ also corresponds to patient's respiratory effort pressure Pₘᵤₛ. The target pressure is determined as mentioned above, and the assisting gas is supplied to a patient with the assisting gas pressure Pᵥₑₙₜ corresponding to the patient's respiratory effort pressure Pₘᵤₛ which will change instantaneously and successively.

In addition, the overall system can be prevented from becoming a positive-feedback configuration by calculating the target pressure Pᵢₙ based on the flow-rate difference ΔF as compared with a related-art arrangement that determines the target pressure Pᵢₙ based on only the measured assisting gas flow-rate signal F. This results in a greater stability margin as compared with the related-art arrangement. As a consequence, runaway phenomena can be prevented from occurring even in cases where a disturbance affects, gas-delivery mechanism has fairly big delay, a user cannot set appropriate flow-rate-assist or volume-assist gain, and/or patient's respiratory condition will change successively. This can reduce a patient's excessive load caused by the runaway phenomena.

With the control apparatus of the invention, as mentioned above, the respiratory effort pressure Pₘᵤₛ is calculated based on the flow-rate difference ΔF. As a result, an objective of the proportional assist ventilation arrangement to deliver to a patient's respiratory airway an assisting gas having a gas pressure Pᵥₑₙₜ proportional to the patient's respiratory effort pressure Pₘᵤₛ is realized reliably.

In addition, the stability margin of the overall system can be bigger than the related-art PAV arrangement in which assist gains are multiplied directly by a measured assisting gas flow-rate. This means that the invention can take a configuration, in which runaway phenomena cannot be likely to occur. As a consequence, the control apparatus can prevent runaway phenomena from occurring even in a case where the actual overall system is changed because of the reasons that a disturbance affects, gas-delivery mechanism has fairly big delay, and/or parameters in a patient's respiratory system change successively. Accordingly it is made possible to realize the control means for gas-delivery mechanism to reduce fairly patient's excessive load caused by runaway phenomena.

In the invention, it is preferable that the flow-rate estimation means has a gas-delivery mechanism model obtained by modeling the gas-delivery mechanism, and the assisting gas flow rate \hat{F} to a patient is estimated based on a series of time-courses that the target pressure signal Pᵢₙ is calculated and thereafter transmitted to the gas-delivery mechanism, and then this gas-delivery mechanism consequently delivers the assisting gas having an assisting gas pressure Pᵥₑₙₜ.

According to the invention, the flow-rate estimation means has a model of gas delivery mechanism in which time response of the gas-delivery mechanism is modeled, and estimates assisting gas flow rate \hat{F} to be delivered to a patient, based on a series of time-courses that the target pressure signal Pᵢₙ is provided and thereafter transmitted to the gas-delivery mechanism, and then this gas-delivery mechanism consequently delivers the assisting gas having an assisting gas pressure Pᵥₑₙₜ to the patient.

The estimated flow rate \hat{F} is determined so as to correspond to the time-response characteristics of the gas-delivery mechanism. In the flow-rate estimation means, for example, the flow rate \hat{F} is calculated taking account of the transfer function with time-delay component, and as a result the flow rate \hat{F} can be calculated more precisely. Moreover, with this feature, the invention can reduce a time difference and/or time lag between a patient's inspiratory duration and/or end-time and assisting gas delivering duration and/or ending time of gas delivery by the gas-delivery mechanism. This time difference and/or time lag between the patient and the ventilator is called as an asynchrony. With the feature, the control apparatus reduces additional patient's breathing load.

In the invention, it is preferable that the flow-rate estimation means has measuring means model obtained by modeling a flow-rate measuring means, and a flow-rate to be delivered to a patient's respiratory airway \hat{F} is estimated based on a series of time-courses that the assisting gas having been delivered to a patient's respiratory airway is measured, and then a measuring result of the measuring means is outputted from the measuring means.

According to the invention, the flow rate \hat{F} is estimated so as to correspond to the time-response characteristics of the measuring means. At the flow-rate estimation step, for example, the flow rate \hat{F} is estimated taking account of the transfer function with time-delay component of the measuring means, and as a result the flow rate \hat{F} can be estimated more precisely. Moreover, with the feature, the asynchrony caused by the delay of measuring means can be reduced. Furthermore, with the feature, additional patient's breathing load can be reduced.

In the invention, it is preferable that the control value calculation means determines a first calculation value (K_{FG}·ΔF) which is a product of a predetermined flow-rate gain K_{FG} and the flow-rate difference Δ\hat{F}, and a second calculation value (K_{VG}·ΔF/s) whish is a product of a predetermined volume gain K_{VG} and an integral of ΔF, and

then adds the first calculation value (K_{FG}·ΔF) and the second calculation value (K_{VG}·ΔF/s) to calculate the target pressure Pᵢₙ.

According to the invention, the control value calculation means calculates a first calculation value in which a predetermined flow-rate gain K_{FG} is multiplied by the flow-rate difference Δ\hat{F}:

a second calculation value in which a predetermined volume gain K_{VG} is multiplied by the time-integral signal Δ\hat{F}; and

then calculates an addition of these two calculation values as a target pressure Pᵢₙ.

The signal Δ\hat{F} is a flow-rate difference caused by the patient's respiratory effort pressure Pₘᵤₛ. By calculating the flow-rate difference Δ\hat{F} as mentioned above, the target pressure signal Pᵢₙ corresponding to the patient's respiratory effort pressure Pₘᵤₛ can be calculated. In this configuration, the patient-assisting gas pressure can be amplified so as to be proportional to the patient's respiratory effort pressure Pₘᵤₛ, when two gain values of K_{FG} and K_{VG} would be appropriately adjusted.

For example, when a model of the respiratory system could be determined adequately and then the flow-rate gain K_{FG} and the volume gain K_{VG} are set multiplying by a factor of B against the real patient's respiratory resistance R and his or her elastance E respectively, assisting gas having a pressure (1+B) times the patient's respiratory effort pressure Pₘᵤₛ can be delivered.

Moreover, according to the invention, the allowable flexibility of the selection of these gains should be enlarged because, as mentioned above, the stability of overall system can be fairly improved, and as a consequence, the user can easily select these gains. The flow-rate gain K_{FG} corresponds to a proportional gain of PI control method, for example, and accordingly speed response against the signal of Pₘᵤₛ can be improved with adjusting the flow-rate gain of K_{FG}. On the other hand, the volume gain K_{VG} corresponds to an integral gain of PI control, and with adjusting this gain, the steady state gain of the target pressure Pᵢₙ can be changed.

And also according to the invention, as mentioned above, with using the flow-rate difference Δ\hat{F} multiplied by the flow-rate gain K_{FG} and the volume gain K_{VG}, the assisting gas pressure Pᵥₑₙₜ could be supplied proportionally to time-varying patient's respiratory effort pressure Pₘᵤₛ, and as a result, it can reduce patient's load.

Furthermore, with adjusting these two gains of K_{FG} and K_{VG}, it is possible to prevent the assisting gas pressure Pᵥₑₙₜ from being oscillatory as well as improving speed response against the respiration pressure Pₘᵤₛ.

In the invention, it is preferable that the flow-rate estimation means further comprises a respiratory airway pressure calculation device for calculating patient's respiratory airway pressure \hat{P}_{aw}, and

the respiratory system model includes:

a subtracter for subtracting an alveolar pressure \hat{P}ₐₗᵥ induced by elastic lung-recoil pressure from the respiratory airway pressure \hat{P}_{aw} calculated by the respiratory airway pressure calculation device when a patient's respiratory effort pressure Pₘᵤₛ does not exist;

an estimated flow-rate calculation device for estimating a flow rate \hat{F} of the assisting gas to be delivered to the patient's respiratory airway by dividing the subtracted value obtained by the subtracter by an estimated patient's respiratory resistance \hat{R};

an assisting gas volume calculation device for calculating a volume \hat{V} of the assisting gas to be delivered to the patient's respiratory airway by integrating successively the flow rate of the assisting gas \hat{F} from starting time of delivering the assisting gas; and

an alveolar pressure calculation device for calculating alveolar pressure \hat{P}ₐₗᵥ by multiplying the calculated volume \hat{V} of the assisting gas by an estimated respiratory elastance Ê to supply the calculated alveolar pressure \hat{P}ₐₗᵥ to the subtracter.

According to the invention, a feature of another configuration for an estimated flow-rate calculation device is provided. It has a calculation device for calculating a patient's respiratory airway pressure signal \hat{P}_{aw} based on an input signal of the target pressure signal Pᵢₙ in the case where a patient's respiratory effort pressure Pₘᵤₛ does not exist. And it has an equivalent model of a patient's respiratory system composed of a subtracter for subtracting a calculated alveolar pressure signal \hat{P}ₐₗᵥ induced by elastic lung-recoil pressure from the predetermined signal \hat{P}_{aw}; an estimated flow-rate calculation device in which an assisting gas flow-rate signal \hat{F} presumed to be delivered to a patient is calculated as the difference divided by an estimated patient's respiratory resistance \hat{R}; an assisted gas volume calculation device in which the estimated assisting gas flow-rate-signal \hat{F} is successively integrated from starting time of each inspiration to calculate an assisted gas volume \hat{V}; and the estimation signal \hat{P}ₐₗᵥ is consequently calculated by multiplying the predetermined signal \hat{V} by estimated respiratory elastance Ê.

The respiratory airway pressure calculation device calculates patient's respiratory airway pressure signal \hat{P}_{aw} based on the input signal of the target pressure Pᵢₙ, and using its signal \hat{P}_{aw}, the assisting gas flow-rate signal \hat{F} is estimated. When the transient response of the gas-delivery mechanism, a delay response characteristics of the pressure measuring means, a calculating delay of the control device, and other characteristics not including overall model could be considered with using this calculation device of \hat{P}_{aw}, the more accurate estimation signal of patient's assisting gas flow rate \hat{F} can be determined.

Moreover, using several calculation devices and the subtracter mentioned above, the assisting gas flow-rate estimation signal \hat{F} is calculated by subtracting the alveolar pressure signal \hat{P}ₐₗᵥ from the patient's respiratory airway pressure signal \hat{P}_{aw} and then this subtracted signal divides by the estimated, or more strictly saying, identified respiratory resistance value \hat{R}. In addition to this calculation, the alveolar pressure signal \hat{P}ₐₗᵥ is calculated by pre-calculating patient's assisted gas volume estimating signal \hat{V} by integration of the assisting gas flow-rate estimation signal \hat{F}, and after by multiplying this volume signal \hat{V} by the estimated respiratory elastance Ê. With these kinds of calculations, a model of patient's respiratory system can be realized precisely.

As long as, for example, a relationship |\hat{R}·s+Ê|<|R·s÷E| is satisfied, the overall system including patient's respiratory system, gas-delivery mechanism in the mechanical ventilator and its control system can be certainly configured as a negative feedback system. With this configuration, the stability margin can be fairly enlarged as compared with the related art. In this equation furthermore, \hat{R} is an estimated, or identified patient's respiratory resistance and Ê is an estimated, or identified respiratory elastance, and R is an real patient's respiratory resistance and also E is real patient's respiratory elastance, and s is Laplace transform operator.

The assisting gas flow-rate estimation signal \hat{F} could be precisely obtained by determining the patient's respiratory airway pressure signal \hat{P}_{aw} using the respiratory airway pressure calculation device. For example, since the respiratory airway pressure calculation device calculates the pressure \hat{P}_{aw} in the respiratory airway in consideration of several kinds of response delay of detectors in the mechanical ventilator, calculation delays in the control device, and other response characteristics not including in the pre-determined respiratory system model, the asynchrony between patient's respiratory timing and the operation of the mechanical ventilator can be prevented. The patient's respiratory model can be made with the subtracter, the estimated flow-rate calculation device and the assisted gas volume calculation device, and within this model, the estimated resistance \hat{R} and estimated elastance Ê should be set.

In the invention, it is preferable that the estimated patient's respiratory resistance \hat{R} is a sum of a first resistance coefficient \hat{R}_{T} which is constant regardless of a flow rate of the assisting gas, and a second resistance coefficient \hat{K}_{T} which is based on the flow rate \hat{F} of the assisting gas calculated by the estimated flow-rate calculation device, and

the estimated respiratory elastance Ê is a value based on the volume \hat{V} of the assisting gas calculated by the assisting gas volume calculation device.

According to the invention, the estimated patient's respiratory resistance \hat{R} can be of a nonlinear characteristic of an addition of two resistance coefficients. A first resistance coefficient is constant and a second resistance coefficient is calculated as a function of the estimated flow-rate signal \hat{F} itself. Also, the estimated patient's respiratory elastance Ê has non-linear characteristics, which is a function of estimated assisted gas volume signal \hat{V}.

In the invention, as mentioned above the estimated patient's respiratory resistance \hat{R} can be set as the additional calculation of the first coefficient and the second coefficient, and the estimated patient's respiratory elastance Ê can be set as a function of estimated assisted gas volume signal \hat{V}. As a consequence, more accurate or more realistic model of the patient's respiratory system in the flow-rate estimation means can be realized, and as a result, the target pressure signal Pᵢₙ can be more accurately proportional to the patient's respiratory effort pressure Pₘᵤₛ.

The estimated resistance \hat{R} and the estimated elastance Ê can be set as variables which represent a real patient's respiratory system. For example, the estimated resistance \hat{R} can be determined in accordance with Roehl's equation and the estimated elastance Ê can be determined in accordance with a hysterics and/or saturated characteristics of patient's respiratory compliance, which is defined as an inverse variable of elastance.

As mentioned above, in the invention, the estimated patient's respiratory resistance \hat{R} can be set as the additional calculation of the first resistance coefficient and the second resistance coefficient, and estimated patient's respiratory elastance Ê can be set as a function of estimated assisted gas volume signal \hat{V}. As a result, the more accurately determined assisting gas pressure Pᵥₑₙₜ can be obtained and this fact may contribute to reduce patient's respiratory load.

In the invention, it is preferable that the control apparatus further comprises modifying means for modifying at least one of the estimated patient's respiratory resistance \hat{R} and the estimated respiratory elastance Ê, based on either the flow rate F of the assisting gas having been delivered to the patient's respiratory airway or an input value inputted from an outside.

According to the invention, modifying means modifies the estimated patient's respiratory resistance \hat{R} or the estimated patient's respiratory elastance Ê in accordance with an input value, which is calculated by an actual flow-rate signal F of a patient's assisted flow delivered to his or her respiratory airway or another input signal set from an outside, for example, using a man-machine interface. The estimated patient's respiratory resistance \hat{R} and/or the estimated patient's respiratory elastance Ê can be set so as to be changeable during control operation, and this capability contributes to improvement of user's convenience. For example actually, these variables can be changed in accordance with influences of a patient's respiratory conditions and/or types of gas-delivery mechanism in a mechanical ventilator, and thereby adaptation of the patient's respiratory model to changes in respiratory resistance and elastance of a real respiratory system can be realized.

Moreover, for example, in the case where the assisting gas flow would be oscillatory, this kind of phenomena can be prevented by reducing the flow gain K_{FG}, and as a consequence, the patient's respiratory load will be reduced.

In the invention, it is preferable that the control apparatus further comprises pressure measuring means for measuring the assisting gas pressure Pᵥₑₙₜ, and

the flow-rate estimation means estimates a flow rate \hat{F} of the assisting gas to be delivered to the patient's respiratory airway based on the assisting gas pressure Pᵥₑₙₜ measured by the pressure measuring means.

According to the invention, an actual signal of pressure measuring means for measuring the assisting gas pressure Pᵥₑₙₜ can be induced, and the assisting gas flow-rate estimation means uses this signal to estimate the assisting gas flow-rate \hat{F}.

In another saying, a more accurate assisting gas pressure Pᵥₑₙₜ can be obtained using the actual pressure detector signal of the assisting gas pressure Pᵥₑₙₜ without any pre-test to get time response of the gas-delivery mechanism. As a consequence, a more accurate estimating signal of patient's respiratory effort pressure Pₘᵤₛ can be obtained in the control apparatus.

The invention provides a patient's respiratory effort pressure estimation apparatus for estimating patient's respiratory effort pressure Pₘᵤₛ when an assisting gas containing oxygen is delivered to a patient's respiratory airway with a predetermined assisting gas pressure Pᵥₑₙₜ, comprising:

flow-rate measuring means for measuring a flow rate F of the assisting gas having been delivered to the patient's respiratory airway;

flow-rate estimation means having a respiratory system model obtained by modeling a patient's respiratory system, for estimating a flow rate \hat{F} of the assisting gas to be delivered to the patient's respiratory airway when the assisting gas is delivered thereto with the assisting gas pressure Pᵥₑₙₜ;

difference calculation means for calculating a flow-rate difference ΔF between the measured flow rate F and the estimated flow rate \hat{F}; and

respiratory effort pressure estimation means for estimating the patient's respiratory effort pressure Pₘᵤₛ based on the flow-rate difference ΔF.

According to the invention, the estimation apparatus estimates patient's respiratory effort pressure Pₘᵤₛ when a gas having a pre-determined assisting gas pressure Pᵥₑₙₜ is delivered to the patient's respiratory airway. This estimation apparatus comprises flow-rate measuring means for measuring an assisting gas flow-rate to a patient's respiratory airway; flow-rate estimation means having a respiratory system model presenting patients' respiratory dynamics, for estimating a flow-rate \hat{F} of an assisting gas to be delivered to the patient's respiratory airway in delivering an assisting gas having an assisting gas pressure Pᵥₑₙₜ; a flow-rate difference calculation means for calculating a flow-rate difference ΔF between the measured flow-rate F and the estimated flow-rate \hat{F} mentioned above, respectively; and patient's respiratory effort pressure estimation means for estimating patient's respiratory effort pressure Pₘᵤₛ based on the flow-rate difference ΔF.

The actual patient's flow-rate F is measured and also assisting gas flow-rate-estimation value \hat{F} is calculated by the estimation means. The measured flow-rate F will be affected by the patient's respiratory effort pressure Pₘᵤₛ but the estimated \hat{F} is not affected thereby.

The flow-rate differential signal ΔF calculated by the flow-rate difference calculating unit represents therefore patient's respiration pressure Pₘᵤₛ. The estimating method of the patient's respiratory effort pressure Pₘᵤₛ can estimate un-measurable patient's respiration pressure Pₘᵤₛ in accordance with using the flow-rate differential signal ΔF. As a consequence, patient's respiration pressure Pₘᵤₛ can be estimated without any invasive measures, and the guidance information, for example, some kinds of information of patients' respiratory system can be presented to persons engaged in medical affairs.

According to the invention, patient's respiration pressure Pₘᵤₛ can be estimated and the estimated Pₘᵤₛ signal, for example, can be used to indicate graphically to persons engaged in medical affairs in order for the persons to confirm patient's respiratory conditions without any invasive measures. In other words, the information of the patient's respiration pressure Pₘᵤₛ can be transferred to the persons to take an adequate medical procedure. Also, the signal can be used for more adequate control means for a gas-delivery mechanism of a mechanical ventilator.

### BRIEF DESCRIPTION OF THE DRAWINGS

Other and further objects, features, and advantages of the invention will be more explicit from the following detailed description taken with reference to the drawings wherein:

FIG. 1 is a schematic diagram showing a configuration of a mechanical ventilator according to an embodiment of the invention;

FIG. 2 is an overall schematic diagram showing a relative configuration between the mechanical ventilator and a patient's respiratory system;

FIG. 3 is a block diagram showing an overall system according to the embodiment of the invention;

FIG. 4 is a graphical representation of a relation between spontaneously breathing gas volume and assisted breathing gas volume in accordance with the overall system 14;

FIG. 5 is an equivalent block diagram mathematically converted from the overall system of the invention shown in FIG. 2;

FIG. 6 is an equivalent block diagram of an overall system of a related art shown in FIG. 24;

FIG. 7 is a graphical representation of stability degree of a system shown in Nyquist diagram;

FIG. 8 is a Nyquist diagram for the overall system in the case of the inequality a<1 of the invention;

FIG. 9 is a Nyquist diagram for the overall system in the case of the inequality a<1 of the related art;

FIG. 10 is a Nyquist diagram for the overall system in the case of the inequality a>1 of the invention;

FIG. 11 is a Nyquist diagram for the overall system in the case of the inequality a>1 of the related-art;

FIG. 12 is a block diagram representing a respiratory airway pressure calculation device;

FIG. 13 is a block diagram showing control value calculation means;

FIG. 14 is an example of dynamic simulation results for the overall system of the preferred embodiment of the invention;

FIG. 15 is an example of dynamic simulation results for the overall system of the related-art;

FIG. 16 is a dynamical simulation result in the case where an amplifying gain β shown in Table 1 is changed into 19;

FIG. 17 is a dynamical simulation result in the case that a flow-amplifying gain β_{FG} would change 50% of the value corresponding to FIG. 16 by a flow-rate gain multiplier;

FIG. 18 is a schematic diagram of an example of the mechanical ventilator;

FIG. 19 is an operational flowchart of a control apparatus main body;

FIG. 20 is a block diagram showing an overall system according to another embodiment of the invention;

FIG. 21 is a block diagram showing an overall system according to still another embodiment of the invention;

FIG. 22 is a block diagram showing an overall system according to further still another embodiment of the invention.

FIG. 23 is a block diagram showing an overall system according to further still another embodiment of the invention;

FIG. 24 is a schematic graph representing the characteristics of patient's respiratory resistance R;

FIG. 25 is a schematic graph explaining the characteristics of patient's respiratory compliance;

FIG. 26 is a block diagram of the overall system of a patient and a mechanical ventilator of a related art; and

FIG. 27 is a graphical representation of a relation between spontaneously breathing gas volume Vₘᵤₛ and assisted breathing gas volume Vₐₛₜ in accordance with the overall system of a related art.

### DETAILED DESCRIPTION

Now referring to the drawings, preferred embodiments of the invention are described below.

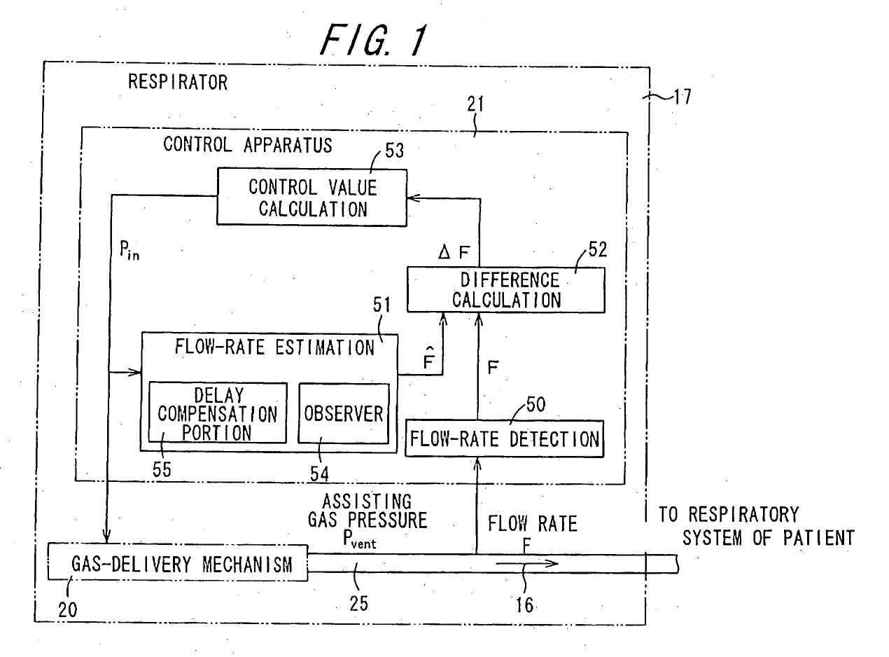

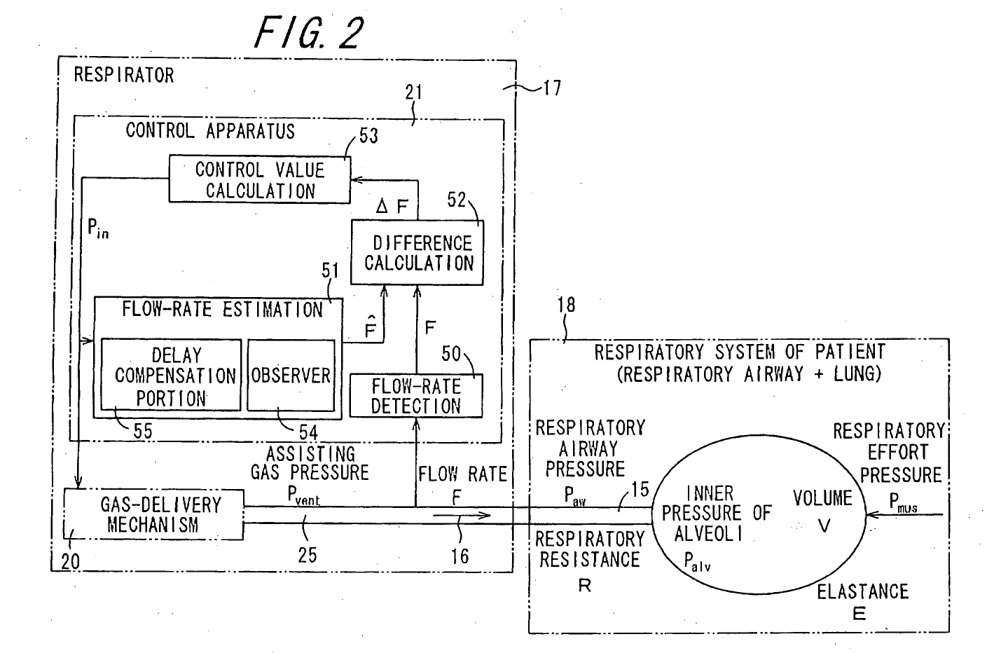

FIG. 1 is a schematic diagram of representing a configuration of a mechanical ventilator 17 as preferred embodiment of the invention. FIG. 2 is an overall schematic diagram indicating a relative configuration between the mechanical ventilator 17 and a patient's respiratory system 18. The mechanical ventilator 17 is composed of a gas-delivery mechanism 20 and a control apparatus 21 for the gas-delivery mechanism 20. The gas-delivery mechanism 20 delivers assisting gas 16 of air or oxygen-enriched air to a patient's respiratory airway 15. The assisting gas 16, for example, is adequately pressurized atmospheric air or oxygen-enriched air. Also, the gas-delivery mechanism is, for another example, the gas-supplying unit such as a pump or high pressure line and is possible to control assisting gas pressure.

A related-art called as Proportional Assist Ventilation method (simply called as PAV method) exists to control the gas-delivery mechanism 20 during patient's inspiratory period when the patient 18 can inspire spontaneously. The control device 21 of an embodiment of the invention controls the gas-delivery mechanism 20 in accordance with the important purpose of the related-art PAV method. The gas-delivery mechanism 20 delivers assisting gas 16 to patient's respiratory airway 15 with assisting gas pressure Pᵥₑₙₜ proportional to patient's respiratory effort pressure Pₘᵤₛ. The patient's respiratory effort pressure Pₘᵤₛ is overall pressure induced by respiratory muscles such as patient's diaphragm and is acted to the patient's respiratory system. In the embodiment of the invention, assisting gas pressure Pᵥₑₙₜ is regarded approximately equal to delivering pressure of the gas-delivery mechanism 20.

The gas-delivery mechanism 20 in the related-art PAV method can deliver gas pressure by the action in accordance with that higher pressure will be delivered when the patient 18 sill inspire much stronger the assisting gas 16, on the contrary lower pressure will be delivered when the patient 18 will inspire weaker the assisting gas 16, and will stop delivering assisting gas 16 when the patient will stop his or her inspiratory effort.

As the result of controlling the gas-delivery mechanism 20 mentioned above, the assisting gas 16 can be delivered gas pressure in accordance with the inspiratory effort of the patient 18, and as a consequence, respiratory load of the patient 18 can be reduced.

The control device 21 calculates target pressure Pᵢₙ so as to correspond to patient's respiration pressure Pₘᵤₛ and then transmits this target pressure Pᵢₙ to the gas-delivery mechanism 20. The gas-delivery mechanism 20 with the given target pressure Pᵢₙ can deliver the assisting gas 16 to the patient 18 in accordance with patient's respiration pressure Pₘᵤₛ.

In this mode of embodiment, the transfer function with the addition of [(s)] represents a transfer function in a manner of Laplace transformation in the complex variable domain of “s”, and the transfer function with the addition of [(jω)] a transfer function in a frequency domain. A value with the addition of [ˆ] represents not an actual value but an estimated value or a calculated value, and [s] a Laplace operator.

The control apparatus 21 includes flow-rate measuring means 50, flow-rate estimation means 51, difference calculation means 52, and control value calculation means 53. The flow-rate measuring means 50 is adapted to detect a flow-rate F of an assisting gas 16 supplied practically into the respiratory airway 15 of a patient. A flow-rate measured by the flow-rate measuring means 50 will hereinafter be referred to as a measured flow-rate F. The measured flow-rate F is a flow-rate of a gas flowing from a gas-delivery mechanism 20 into an inspiratory conduit 25, and handled to be equal or approximate to that of a gas flowing in the respiratory airway of the patient. Since this measured flow-rate F varies depending upon the influence a respiratory effort pressure Pₘᵤₛ, the measured quantity becomes a flow-rate in a respiratory system with the respiratory effort pressure Pₘᵤₛ added thereto.

The flow-rate measuring means 50 is adapted to measure the flow-rate of the assisting gas 16 flowing in the inspiratory conduit 25. The inspiratory conduit 25 is a conduit for introducing the assisting gas 16 from a pressure source of the gas-delivery mechanism 20 into the respiratory airway of the patient. For example, the flow-rate measuring means 50 is materialized by a differential flow meter. When the flow-rate measuring means 50 measures the flow-rate F of the assisting gas, the same measuring means 50 gives the measured flow-rate F to the difference calculation means 52.

The flow-rate estimation means 51 has a so-called observer 54, a model of a respiratory system obtained by simulating and modeling the respiratory system of the patient. The observer 54 is adapted to calculate a flow-rate \hat{F}, which will be supplied to the patient when the respiratory effort pressure Pₘᵤₛ does not exist, on the basis of the information corresponding to a target pressure Pᵢₙ calculated by the control value calculation means 53.

The flow-rate estimated by the flow-rate estimation means 51 will hereinafter be referred to as an estimated flow-rate \hat{F}. The estimated flow-rate \hat{F} becomes the flow-rate in the respiratory system at the calculated value \hat{P}aw of the respiratory airway pressure, which corresponds to the assisting gas pressure Pᵥₑₙₜ. When the flow-rate estimation means 51 estimates the flow-rate \hat{F} of the assisting gas, the estimation means gives a signal representative of the estimated flow-rate to the difference calculation means 52.

The difference calculation means 52 calculates a flow-rate difference ΔF, which becomes a value obtained by subtracting the estimated flow-rate \hat{F} from the measured flow rate F, and gives the calculation results to the control value calculation means 53. The control value calculation means 53 adds gain, which is set in advance, to the flow-rate difference ΔF, and calculates the target pressure Pᵢₙ relative to the assisting gas pressure Pᵥₑₙₜ.

The control value calculation means 53 gives a signal representative of the calculated target pressure Pᵢₙ to the flow-rate estimation means 51 and gas-delivery mechanism 20 respectively. The gas-delivery mechanism 20 supplies the assisting gas 16 to the respiratory airway 15 of the patient with the discharge pressure, i.e. the assisting gas pressure Pᵥₑₙₜ based on the signal representative of the target pressure Pᵢₙ given from the control value calculation means 53. The flow-rate estimation means 51 calculates in order the estimated flow-rate \hat{F} on the basis of a signal representative of the target pressure Pᵢₙ given from the control value calculation means 53.

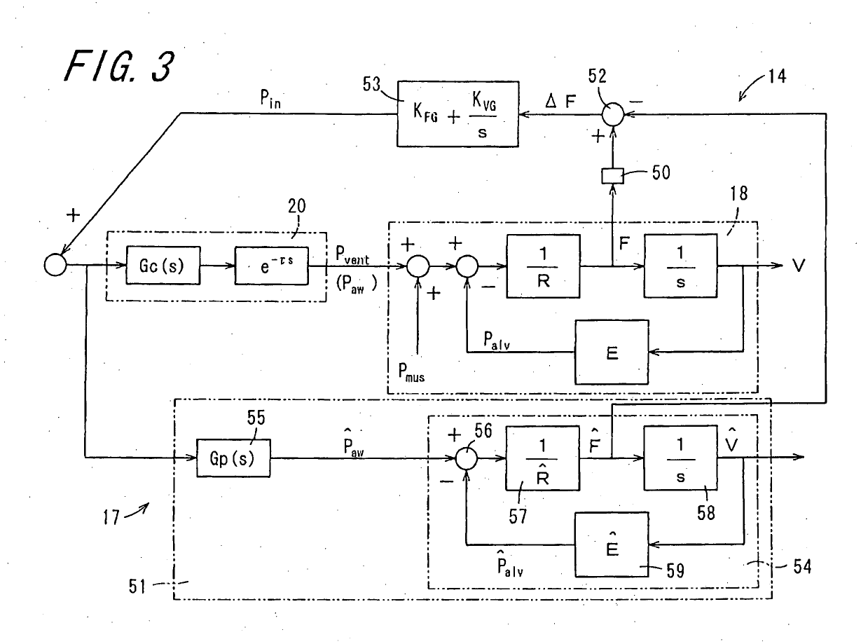

FIG. 3 is a block diagram concretely showing the whole system of a mode of embodiment of the invention. The flow-rate estimation means 51 further has a delay compensation portion 55 in addition to the observer 54. The delay compensation portion 55 compensates first-order delay factors of each structural part constituting the overall system 14, such as a delay factor of, for example, the gas-delivery mechanism 20 and a delay factor of the air circuit, and a dead time factor. In this mode of embodiment, the delay compensation portion 55 corresponds to the respiratory airway pressure calculation device defined in Claims.

The respiratory airway pressure calculation device 55 calculates the respiratory airway pressure \hat{P}_{aw} of the patient 18 on the basis of the target pressure Pᵢₙ. The respiratory airway pressure of the patient calculated by the respiratory airway pressure calculation device 55 will hereinafter be referred to as the calculated respiratory airway pressure \hat{P}_{aw}, and the actual respiratory airway pressure simply as the respiratory airway pressure P_{aw}. The respiratory airway pressure calculation device 55 gives a signal representative of the calculated respiratory airway pressure \hat{P}_{aw} to the observer 54.

The observer 54 estimates the estimated flow-rate \hat{F} of the assisting gas in a case where the assisting gas is supplied to the respiratory airway 15 of the patient 18 with \hat{P}_{aw}. The observer 54 has a subtracter 56, an estimated flow-rate calculation device 57, an assisting gas volume calculation device 58 and an alveoli pressure calculation device 59.

The subtracter 56 is given a calculated respiratory airway pressure \hat{P}_{aw} from the respiratory airway pressure calculation device 55, and a calculated alveoli pressure calculation device 59. The subtracter 56 takes the calculated alveoli pressure \hat{P}ₐₗᵥ from the calculated respiratory airway pressure \hat{P}_{aw}, and gives the resultant value to the estimated flow-rate calculation device 57. The calculated alveoli pressure Pₐₗᵥ will be described later.

The estimated flow-rate calculation device 57 divides a subtracted value obtained by the subtracter 56 by an estimated respiratory resistance \hat{R}, and the resultant value is calculated as an estimated flow-rate \hat{F}.

The estimated respiratory resistance \hat{R} is a value determined by estimating the respiratory resistance of the patient, and set in advance by, for example, a person relating to the medical care. The estimated respiratory resistance \hat{R} may be set in advance by a value measured by a measuring instrument. As will be described later, the estimated respiratory resistance \hat{R} in the overall system 14 according to the invention may be set not accurately in agreement with the actual respiratory resistance R of the patient.

The assisting gas volume calculation device 58 integrates in order the estimated flow-rate \hat{F} calculated by the estimated flow-rate calculation device 57 from the assisting gas supply starting time, and the integrated value is calculated as the volume \hat{V} of the assisting gas. The assisting gas volume calculation device 58 serves as a so-called integrator. The volume of the assisting gas calculated by the assisting gas volume calculation device 58 will hereinafter be referred to as \hat{V}, and distinguished from the actual volume V of the assisting gas in some cases.

The alveoli pressure calculation device 59 multiplies the calculated volume \hat{V} by the estimated elastance Ê of the lung set in advance, and the resultant value is calculated as the calculated alveoli pressure \hat{P}ₐₗᵥ. The alveoli pressure calculation device 59 gives the calculated alveoli pressure \hat{P}ₐₗᵥ to the subtracter 56. The calculated alveoli pressure \hat{P}ₐₗᵥ is a value obtained by estimating the pressure in the alveoli, and referred to by distinguishing the same from the actual alveoli pressure Pₐₗᵥ.

The estimated elastance Ê of the lung is a value obtained by estimating the elastance of the lung of the patient, and set in advance by a person relating the medical care. The elastance Ê to be estimated of the lung may be set in advance by a value measured by a measuring instrument, such as a ventilation dynamic inspection apparatus. As will be described later, in the whole system 14 according to the invention, the elastance Ê of the lung may not be set accurately in agreement with the actual elastance E of the lung of the patient.

When the assisting gas flows in the respiratory airway, a pressure loss substantially proportional to the flow-rate F of the assisting gas occurs, and the pressure in the lung becomes lower than that in the respiratory airway. The respiratory resistance R represents the relation between the flow-rate F of this assisting gas and pressure loss. A value (F·R) obtained by multiplying the flow-rate F of the assisting gas by the respiratory resistance R becomes a loss pressure caused by the resistance of the respiratory airway. For example, a general resistance R of the respiratory airway is 5 to 30 (cmH₂O)/(liter/second). However, the respiratory airway pressure R varies greatly depending upon the condition of the patient.

When the assisting gas is supplied into the lung, the inner pressure Pₐₗᵥ of the alveoli increases substantially in proportion to an increase in the volume V of the assisting gas supplied into the lung. The elastance E of the lung represents the relation between the volume V of the assisting gas and the inner pressure Pₐₗᵥ of the alveoli. A value (V·E) obtained by multiplying the volume V of the assisting gas by the elastance of the lung comes to represent the inner pressure Pₐₗᵥ of the lung. This inner pressure Pₐₗᵥ of the alveoli becomes a pressure against the influx of the assisting gas. For example, the elastance E of a general lung is 1/20 to 1/50 (milliliter)/(cmH₂O). However, the elastance E of the lung varies depending upon the condition of the patient.

On the basis of such characteristics of the respiratory system, a respiratory system model represented by the observer 54 is obtained. The respiratory system model represented by the observer 54 is set so as to have the following relation.
\hat{P}_{aw}−\hat{P}ₐₗᵥ=\hat{R}·\hat{F} (1)
∫\hat{F}=\hat{V} (2)
\hat{P}ₐₗᵥ=Ê·\hat{V} (3)

The respiratory system model represented by the observer 54 is a model of the respiratory system of the patient in which the respiratory effort pressure Pₘᵤₛ is set zero. In this model, a value obtained by subtracting the calculated alveoli pressure \hat{P}ₐₗᵥ from the calculated respiratory airway pressure \hat{P}_{aw} is equal to that obtained by multiplying the estimated flow-rate \hat{F} and estimated respiratory resistance \hat{R} together. A value obtained by integrating the estimated flow-rate \hat{F} from the assisting gas supply starting time is equal to that of the calculated volume \hat{V}. The calculated alveoli pressure \hat{P}ₐₗᵥ is equal to a value obtained by multiplying the estimated elastance Ê of the lung and calculated volume \hat{V} together.

Therefore, when the calculated respiratory airway pressure \hat{P}_{aw} is set as an input value with the estimated flow-rate \hat{F} as an output value, the transfer function G(s)₅₄ of the observer 54 is as follows.

$$\frac{s}{\hat{R} \cdot s + \hat{E}} \qquad ( 4 )$$

In this expression, \hat{R} represents estimated respiratory resistance; and Ê estimated elastance of the lung. In other equations and expressions, the symbols shown in the above expression (4) represent the same meanings. Such a respiratory system model represented by the observer 54 is an example of embodiment, and may be made of other model formed by simulating the respiratory system of the patient.

The control value calculation means 53 determines a first calculated value (K_{FG}·ΔF) obtained by multiplying the flow-rate difference ΔF which is calculated by the difference calculation means 52, by a preset factor of flow-rate gain K_{FG}, and a second calculated value (K_{VG}·ΔF/s) obtained by multiplying a value in which the flow-rate differences ΔF are sequentially accumulated from the assisting gas supply starting time, by a preset factor of volume gain K_{VG}. The target pressure Pᵢₙ relating to the assisting gas pressure Pᵥₑₙₜ is calculated by summing the first calculated value and second calculated value. When the flow-rate difference ΔF is set as an input value with the target pressure Pᵢₙ as an output value, the transfer function G(s)₅₃ of the control value calculation means 53 is as shown below.

$${K}_{FG} + \frac{{K}_{VG}}{s} \qquad ( 5 )$$

In this expression, K_{FG} represents the flow-rate gain, and K_{VG} the volume gain. In other expressions, the symbols shown in the above expression represent the same meanings. For example, the flow-rate gain K_{FG} is set to a value (\hat{R}·β_{FG}) obtained by multiplying the estimated respiratory resistance \hat{R} by preset the flow rate amplification gain β_{FG}, and the volume gain K_{VG} is set to a value (Ê·β_{VG}) obtained by multiplying the elastance Ê of the lung by the preset volume amplification gain β_{VG}. When the flow-rate amplification gain β_{FG} and the volume amplification gain β_{VG} are set to the same value, these gains will hereinafter be referred simply as amplification gain β. The amplification gain β in a case where \hat{R}=R and Ê=E is expressed by B.

Although the flow-rate estimation means 51, difference calculation means 52, and control value calculation means 53 are described separately for the purpose of easy understanding, they may be described in the form of transfer functions in which equivalent conversions are made. The flow-rate estimation means 51, difference calculation means 52 and control value calculation means 53 may be realized by executing a predetermined operation program with a numerical value computable computer.

In the mode of embodiment of the invention, the transfer functions of the gas-delivery mechanism 20 include a dead time factor. FIG. 3 illustrates separately transfer functions Gc(s) from which the time element is removed and transfer functions e^{-τ·s} of the dead time factor, which are among the transfer functions of the gas-delivery mechanism 20. The transfer function G(s)₂₀ in a case where the target pressure Pᵢₙ is set as an input value with the discharge pressure Pᵥₑₙₜ as an output value is shown below.
Gc(s)·e^{-τ·s} (6)

In this expression, Gc(s) represents the transfer function of the gas-delivery mechanism 20 from which the dead time factor is removed. The e represents naturalized logarithm, and τ the dead time needed from the time at which the target pressure Pᵢₙ is given to that at which the gas-delivery mechanism 20 starts regulating the assisting gas pressure Pᵥₑₙₜ. In the other expressions, the symbols shown in this expression indicate the same meanings.

The actual respiratory system of the patient is different from the respiratory system model of the observer 54 in that the respiratory effort pressure Pₘᵤₛ is given to the former in addition to the assisting gas pressure Pᵥₑₙₜ. In the mode of the embodiment of the invention, a pressure loss in the inspiratory conduit is small, so that the assisting gas pressure Pᵥₑₙₜ constituting the discharge pressure of the gas-delivery mechanism 20 and the actual respiratory airway pressure P_{aw} of the patient are handled to be equal or approximate to each other.

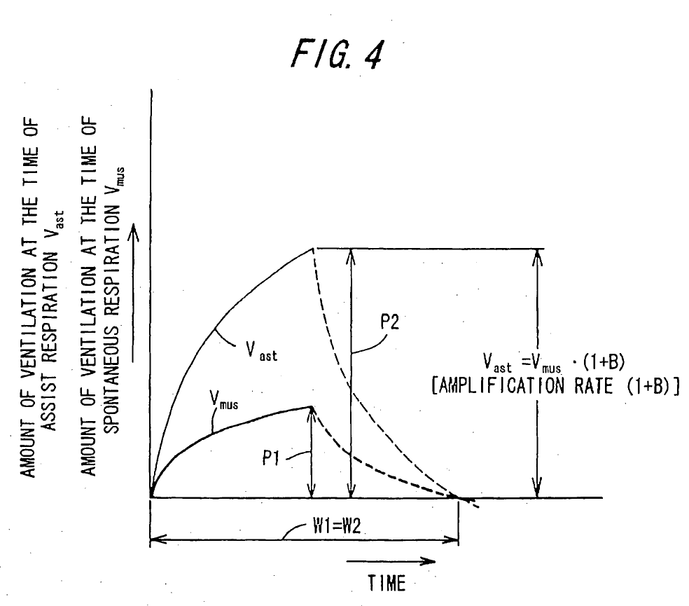

FIG. 4 is a graph showing the relation between the air ventilation Vₘᵤₛ at the time of spontaneous respiration in the whole system 14 according to the invention and that Vₐₛₜ at the time of assist respiration. The assisting gas pressure Pᵥₑₙₜ is amplified at an amplification factor (1+B) times as high as that of the respiratory effort pressure Pₘᵤₛ in accordance with the variation with the lapse of time of the respiratory effort pressure Pₘᵤₛ. The air ventilation is equal to the volume of the assisting gas flowing into the lung. The B represents the amplification gain β in a case where \hat{R}=R with Ê=E as mentioned above. The air ventilation Vₐₛₜ at the time of assist respiration by the mechanical ventilator 17 of this mode of embodiment is amplified (1+B) times that Vₘᵤₛ at the time of spontaneous respiration.

When the condition of the patient is shifted from expiratory period to inspiratory period, the patient operates the respiration muscles, such as the diaphragm. As a result, the air ventilation Vₘᵤₛ at the time of spontaneous respiration and respiratory effort pressure Pₘᵤₛ increases gradually with the lapse of time, and decreases gradually when the air ventilation Vₘᵤₛ and respiration pressure Pₘᵤₛ reach certain peaks P1. The condition of the patient is shifted from the inspiratory period to the expiratory period.

In general, the air ventilation Vₘᵤₛ and respiratory effort pressure Pₘᵤₛ of the patient with spontaneous breathing firstly make a gentle gradually increasing curve during the inspiratory period as a waveform with respect to time, and secondly make an acute decreasing curve when the waveform changes from an ultimate level into the expiratory period. However, the air ventilation Vₘᵤₛ and respiratory effort pressure Pₘᵤₛ vary greatly depending upon conditions of patients, and accordingly the peak value P1 and respiration period W1 vary greatly.

The gas-delivery mechanism 20 controlled by the control apparatus 21 discharges the assisting gas at the assisting gas pressure Pᵥₑₙₜ so as to attain the respiratory airway pressure P_{aw} (=Pₘᵤₛ·β) amplified proportionally to the respiratory effort pressure Pₘᵤₛ of the patient and on the basis of the preset amplified gain β. For example, when the peak value P1 of the respiratory effort pressure Pₘᵤₛ is small and the inspiratory period W1 is short, the assisting gas pressure Pᵥₑₙₜ is controlled so that the peak value P2 of the respiratory airway pressure P_{aw} becomes small and the assisting gas supply period W2 becomes short. Similarly, when the peak, value P1 of the respiratory effort pressure Pₘᵤₛ is large and the inspiratory period W1 is long, the assisting gas pressure Pᵥₑₙₜ is controlled so that the peak of the respiratory pressure P_{aw} becomes large and the supply gas supply period W2 becomes long.

According to the control apparatus 21 in the mode of embodiment, the assisting gas pressure Pᵥₑₙₜ is determined on the basis of the flow-rate difference ΔF. Although the measured flow-rate F varies depending upon the respiratory effort pressure Pₘᵤₛ of the patient, the estimated flow-rate \hat{F} does not receive the influence of the respiratory effort pressure Pₘᵤₛ of the patient. Therefore, the flow-rate difference ΔF becomes a value obtained by extracting the variation of the respiratory effort pressure Pₘᵤₛ. This enables estimation of the respiratory effort pressure Pₘᵤₛ which is usually difficult to measure, and as a consequence the estimation of the respiratory effort pressure Pₘᵤₛ serves as a disturbance observer in which the respiratory effort pressure Pₘᵤₛ is regarded as a disturbance.

When the target pressure Pᵢₙ is thus calculated in accordance with the flow-rate difference ΔF relative to the respiratory effort pressure Pₘᵤₛ, the assisting gas can be supplied to the patient with an assisting gas pressure Pᵥₑₙₜ which follows the respiratory effort pressure Pₘᵤₛ at substantially real time.

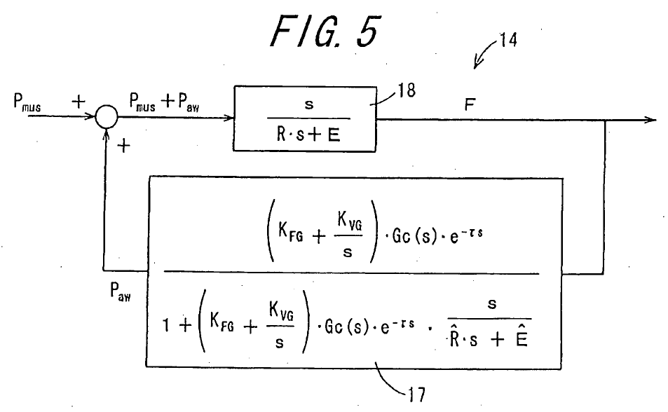

FIG. 5 is a block diagram showing the equivalently converted and arranged overall system 14 of the invention of FIG. 3. When the respiratory effort pressure Pₘᵤₛ is regarded as a set value and a sum of the respiratory effort pressure Pₘᵤₛ and the assisting gas pressure Pᵥₑₙₜ is regarded as an output value in the overall system 14 in the invention, the transfer function thereof is represented by the following expression.

$$\frac{{P}_{mus} + {P}_{vent}}{{P}_{mus}} = 1 + \frac{Gc ( s ) \cdot \frac{{K}_{FG} \cdot s + {K}_{VG}}{R \cdot s + E}}{1 + Gc ( s ) \cdot \frac{{K}_{FG} \cdot s + {K}_{VG}}{R \cdot s + E} \times ( \frac{R \cdot s + E}{\hat{R} \cdot s + \hat{E}} - 1 )} \qquad ( 7 )$$

The symbols in this expression correspond to those mentioned above.

When Gc(s)=1, \hat{R}=R, Ê=E, K_{FG}=\hat{R}·B, and K_{VG}=Ê·B in the overall system 14 in the invention, a pressure (Pₘᵤₛ+Pᵥₑₙₜ) obtained by adding the respiratory effort pressure Pₘᵤₛ and assisting gas pressure Pᵥₑₙₜ together is amplified as (1+B) times as large as the respiratory effort pressure Pₘᵤₛ. When the respiratory resistance R and elastance E can be estimated accurately, the assisting gas pressure Pᵥₑₙₜ can consequently be amplified with respect to the respiratory effort pressure Pₘᵤₛ as long as B is larger than 0.

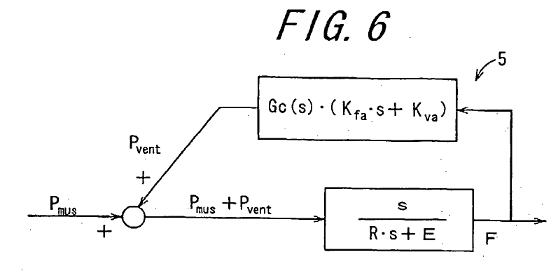

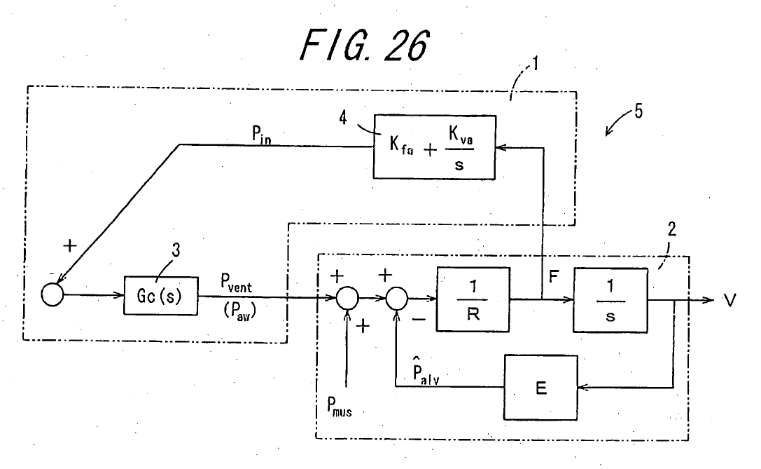

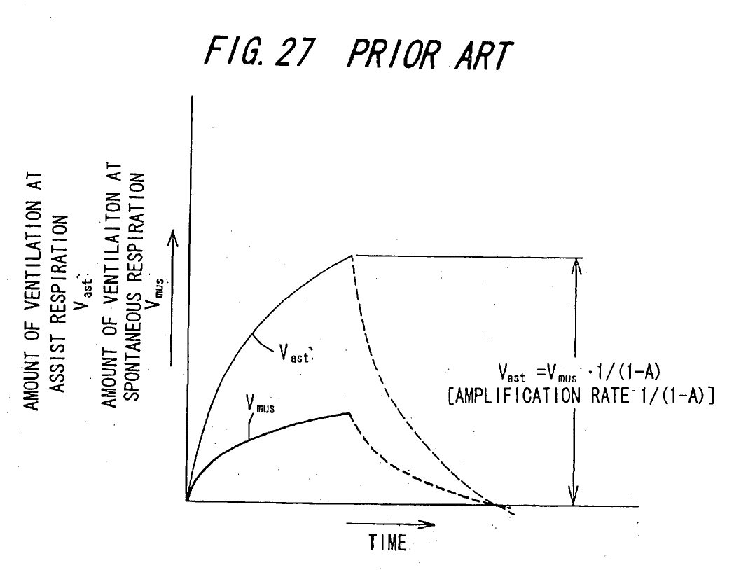

FIG. 6 is a block diagram showing the related art whole system 5 shown in FIG. 26. The transfer function G(s)₅ of the related art overall system 5, in which the respiratory effort pressure Pₘᵤₛ is regarded as a set value and a sum of the respiratory effort pressure Pₘᵤₛ and the assisting gas pressure Pᵥₑₙₜ is regarded as an output value in the related art overall system 5, the transfer function G(s)₅ is shown below.

$$\frac{{P}_{mus} + {P}_{vent}}{{P}_{mus}} = 1 + \frac{Gc ( s ) \cdot \frac{{K}_{fa} \cdot s + {K}_{Va}}{R \cdot s + E}}{1 - Gc ( s ) \cdot \frac{{K}_{fa} \cdot s + {K}_{Va}}{R \cdot s + E}} \qquad ( 8 )$$

In this expression, the symbols correspond to those mentioned above.

When Gc(s)=1, \hat{R}=R, Ê=E, K_{fa}=\hat{R}·A, and K_{Va}=Ê·A in the related art overall system 5, a pressure (Pₘᵤₛ+Pᵥₑₙₜ) obtained by adding the respiratory effort pressure Pₘᵤₛ and assisting gas pressure Pᵥₑₙₜ together is multiplied as 1/(1−A) times as large as the respiratory effort pressure Pₘᵤₛ. When A<0 or A>1 in this case, the respiratory effort pressure Pₘᵤₛ cannot be amplified.

As described above, in the overall system 14 in the invention the amplification of the spontaneous respiratory pressure can be amplified as long as B is larger than 0. In the related art overall system 5, it is necessary that A becomes 0<A<1. Therefore, in the overall system 14 in the invention, the degree of freedom of the selection of gain can be improved.

It is impossible that the estimated respiratory resistance \hat{R} and estimated elastance Ê be set to values completely equal to those of actual respiratory resistance R and actual elastance E. In general, when there is deviation in these values, they are usually (\hat{R}≠R, Ê≠E). Moreover, when it is usual that there is a certain delay in the gas-delivery mechanism materializing the mechanical ventilator 17, (Gc(s)≠1), and such a general case will be described later.

In the related art overall system 5 and overall system 14 in the invention, the transient response thereof becomes unstable depending upon the factors, such as the variation of the condition of the patient, setting errors of parameters and disturbance. When such occurs, the assisting gas of an excessive pressure is supplied to the patient, and runaway occurs in some cases.

However, in the overall system 14 in the invention, a margin for stabilizing the same is large as compared with that of the related art overall system 5 as will be described later. Therefore, even when the variation of the condition of the patient and setting error of the parameters occur, and even when the gain and disturbance are set large and exerted on the overall system respectively, the runaway can be made to rarely occur.

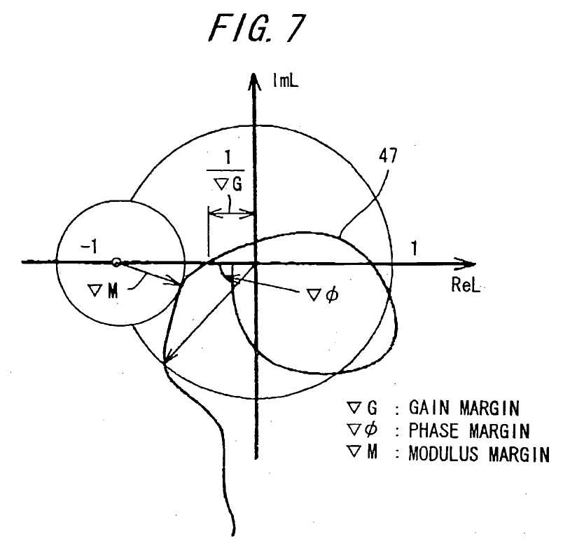

FIG. 7 is a graphical representation for showing the stability margin of a system. In the overall system, a Nyquist diagram is used so as to judge the stability margin, i.e. the difficulty degree of the overall system of being put in an unstable limit condition.

For example, there is one method of judging the stability margin on the basis of a distance ∇M at which the vector locus 47 of a loop transfer function and a stability limit point (−1, 0) become closest to each other. This distance ∇M is called a modulus margin ∇M. The modulus margin ∇M is expressed by the following equation when the loop transfer function is expressed by G_{OL}(jω). It is judged that, the larger the modulus margin ∇M is, the more difficult the overall system becomes unstable.
∇M=|1+G_{OL}(jω)|ₘᵢₙ (9)

A modulus margin ∇My in the related art overall system 5 is represented by the following expression in view of the equation (1).

$$▽ My = {\vert 1 - Gc ( j ω ) \cdot α \cdot \frac{( \hat{R} \cdot j ω + \hat{E} )}{R \cdot j ω + E} \vert}_{min} \qquad ( 10 )$$

The modulus margin ∇Mk in the overall system 14 of one mode of embodiment of the invention is expressed by the following equation in view of the equation (8).

$$▽ Mk = {\vert 1 + Gc ( j ω ) \cdot β \cdot ( 1 - \frac{( \hat{R} \cdot j ω + \hat{E} )}{R \cdot j ω + E} ) \vert}_{min} \qquad ( 11 )$$

In order to compare the equations (10), (11) with each other easily, the items are set to K_{fa}=\hat{R}·α, K_{Va}=Ê·α, K_{FG}=\hat{R}·β, and K_{VG}=Ê·β.

Suppose that the estimated error of the respiratory system in the invention and that thereof in the related art respiratory system are identical with each other with \hat{R}/R=Ê/E=a, the modulus margin ∇M can be expressed by the following equations.
∇My=|1−α·a·Gc(jω)|ₘᵢₙ (12)
∇Mk=|1+(1−a)·β·Gc(jω)|ₘᵢₙ (13)

In these equations, a shows a deviation of the estimated value and actual value from each other, and α and β are values relative to the amplification factor at which the assisting gas pressure Pᵥₑₙₜ is amplified with respect to the respiratory effort pressure Pₘᵤₛ.

The transfer function Gc(jω) generally includes a first-order delay factor and a dead time factor. The transfer function Gc(jω)₂₀ of the gas-delivery mechanism 20 is shown below.

$$\frac{{ⅇ}^{- τ \cdot j ω}}{Tc \cdot j ω + 1} \qquad ( 14 )$$

In this expression, Tc represents time constant of the gas-delivery mechanism 20. The e represents the bottom of the naturalized logarithm, and τ the dead time of the gas-delivery mechanism 20. A Nyquist diagram of the overall system in the case where the gas-delivery mechanism has such a transfer function is shown below.

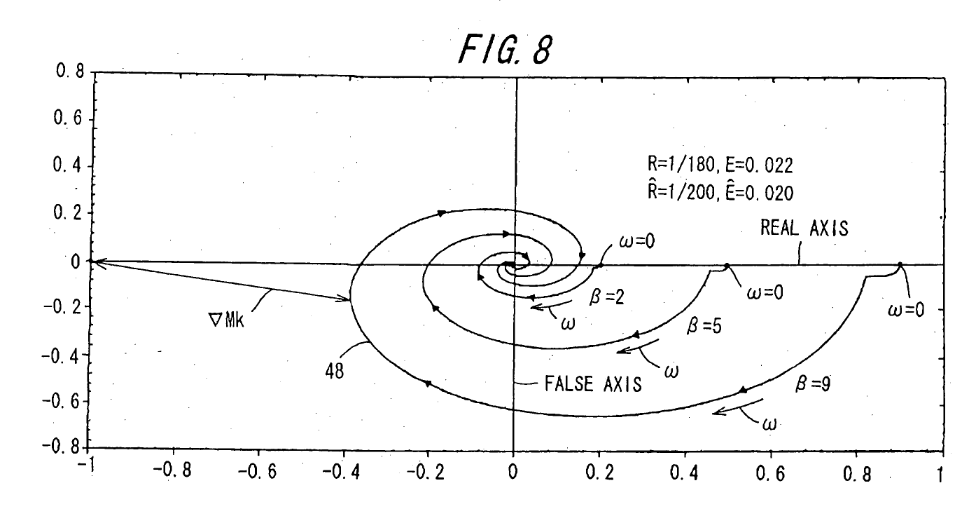

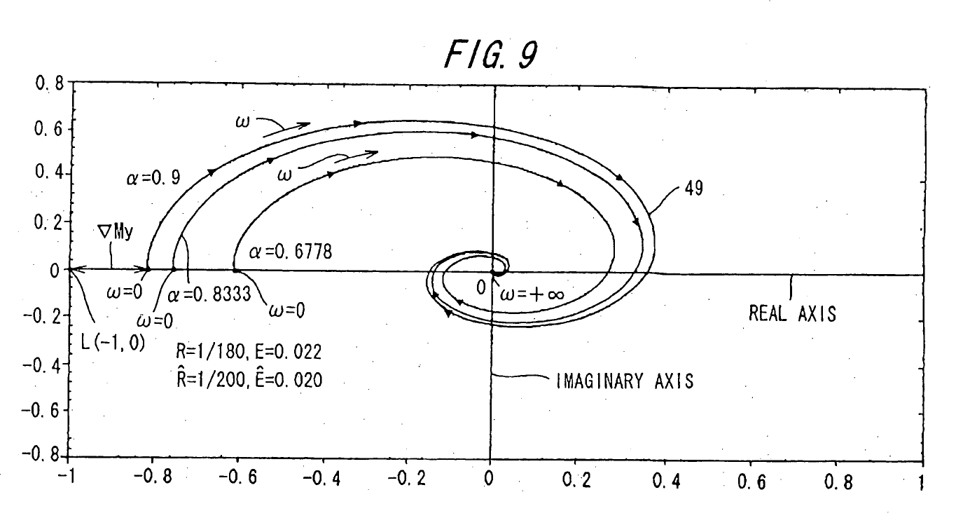

FIG. 8 shows a Nyquist diagram of an overall system 14 according to the invention in which a<1. FIG. 9 shows a related art overall system 5 in which a<1.

In view of the equation (12), it is necessary that α·a becomes 0<α·a<1 in the case of the related art overall system 5. In this case, a vector locus of a loop transfer function has a factor of a transfer function −Gc(jω) of a gas-delivery mechanism 20 of a positive feedback configuration. Owing to this, as an angular frequency ω increases from a zero state, the vector locus is drawn so as to extend from a negative region of a real axis toward an origin O as shown in FIG. 9. Therefore, the vector locus of the related art overall system 5 approaches most a stability limit point L(−1, 0) when the angular frequency ω is zero. As a result, even though the overall system 5 is stable, a modulus margin ∇My is small. When an assist rate α is increased, the overall system 5 is liable to become unstable.

In view of the equation (13), when a<1 in the case of the overall system 14 according to the invention, the vector locus of the loop transfer function has a factor of the transfer function (jω) of the gas-delivery mechanism 20 of a negative feedback configuration. Therefore, as the angular frequency ω increases from a zero state as shown in FIG. 8, the vector locus is drawn so as to extend from a positive region of the real axis toward the origin O. Therefore, the overall system 14 according the invention approaches most the stability limit point L when the angular frequency ω advances beyond zero. As a result, the modulus margin ∇Mk of the overall system 14 according to the invention becomes larger than that of the related art overall system 5. Therefore, even when an amplification gain β is set large, the overall system 14 rarely becomes unstable. To be concrete, as long as |\hat{R}·s+Ê|<|R·s+E| can be set, a negative feedback configuration can be formed necessarily.

For example, the vector locus 48 of the overall system 14 according to the invention of β=9 shown in FIG. 8 and that 49 of the related art overall system 5 of α=0.9 shown in FIG. 9 indicate cases where the amplification factors are set equal. As is clear from FIG. 8 and FIG. 9, it is understood that the modulus margin ∇M of the overall system 14 according to the invention is larger than that of the related art overall system 5, and that the stability margin of the former overall system 14 is larger than that of the latter overall system 5.

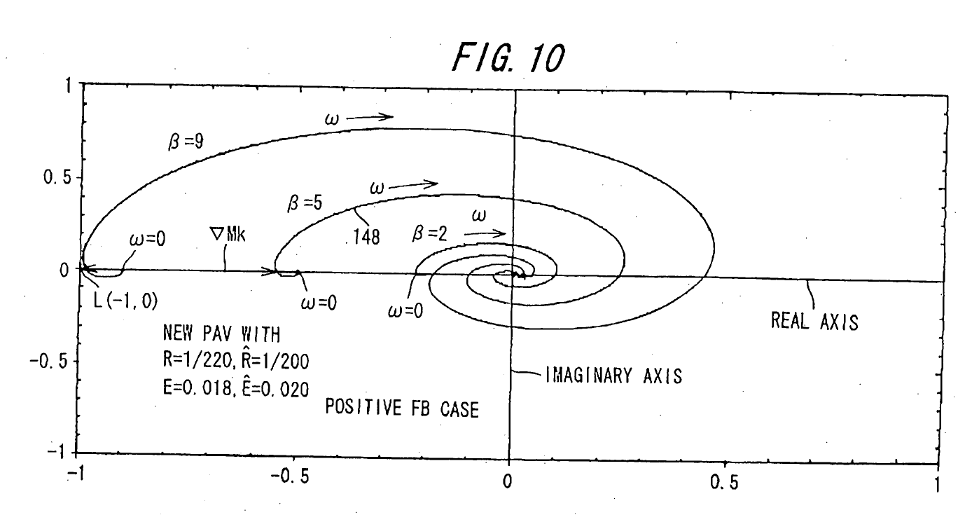

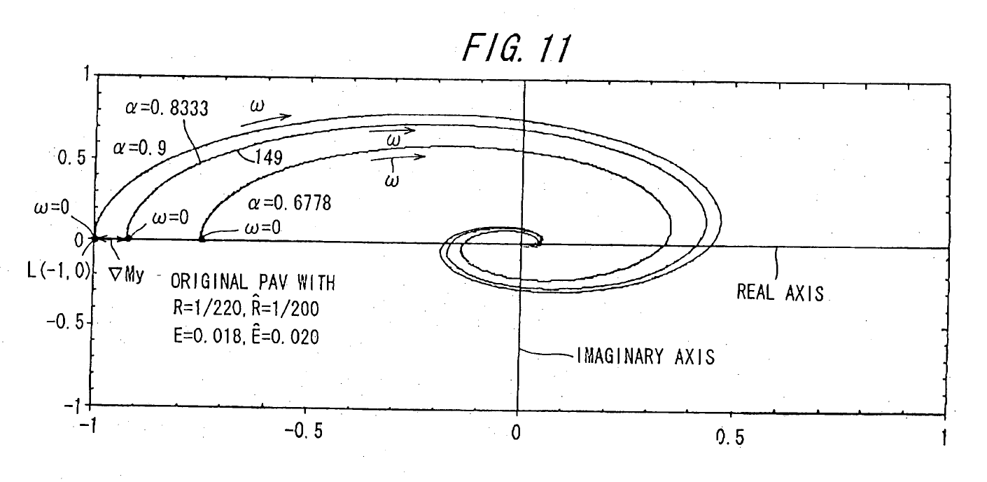

FIG. 10 shows a Nyquist diagram of the overall system 14 according to the invention in which a>1. FIG. 11 shows a Nyquist diagram of the related art overall system 5 in which a>1. When a>1, the overall system 14 has a factor of the transfer function Gc(jω) of a positive feedback configuration. Even in such a case, the modulus margin ∇Mk of the overall system 14 according to the invention is larger than that ∇My of the related art overall system as is clear from the equations (12) and that (13).

For example, the vector locus 148 of the overall system 14 according to the invention of β=5 shown in FIG. 10 and that 149 of the related art overall system 5 of α=0.8333 shown in FIG. 11 indicate cases where the amplification factors are set equal. As is clear from FIG. 10 and FIG. 11, it is understood that the modulus margin ∇M of the overall system 14 according to the invention is larger even though a>1than that of the related art overall system 5, and that the stability margin of the former overall system 14 is larger than that of the latter overall system 5.

As described above, the overall system 14 in the mode of embodiment of the invention can improve the stability margin thereof, and substantially prevent the same overall system 14 from becoming unstable. Namely, even when an actual overall system is different from the overall system 14 simulated by a control apparatus 21 on the basis of setting errors of parameters, variation of the condition of a patient, disturbance and the like, the actual overall system rarely becomes a positive feedback configuration, so that the occurrence of runaway can be prevented. This enables a method of controlling a gas-delivery mechanism in which the patient's load is further reduced.

Even when the estimated respiratory resistance \hat{R} and estimated elastance Ê are shifted slightly from the actual respiratory resistance R and elastance E, the overall system 14 is prevented from becoming unstable since the stability margin of the overall system is large as mentioned above, and the assisting gas pressure Pᵥₑₙₜ can be amplified by regulating the amplification gain β. Especially, as mentioned above, a<1, i.e., \hat{R}<R, Ê<E is obtained, so that a negative feedback configuration is necessarily formed. Therefore, the stability margin can be set larger. Accordingly, when a doctor or therapist and so forth ascertains the condition of a patient and sets an estimated respiratory resistance \hat{R} which becomes \hat{R}<R, Ê<E and an estimated elastance Ê, the possibility that runaway occurs becomes small even when accurate respiratory resistance R and an estimated elastance Ê are not determined, and the gas-delivery mechanism can be controlled.

For example, when the respiratory airway of a patient is clogged up with sputum, which constitutes an example possible to occur in practice, an actual respiratory resistance R becomes larger than a Generally assumed respiratory resistance R. In such a case, R becomes large with respect to \hat{R} in the overall system according to the invention, so that R necessarily changes toward the stabler side.

In the mode of embodiment of the invention, the assisting gas pressure Pᵥₑₙₜ is determined substantially on the basis of a real-time respiratory effort pressure Pₘᵤₛ by calculating a target pressure Pᵢₙ in accordance with a flow-rate difference ΔF. Therefore, an assisting gas pressure Pᵥₑₙₜ based on the actual respiratory effort pressure Pₘᵤₛ of a patient can be given even when the waveform pattern, peak value and generation period thereof vary.

This enables a primary purpose of a proportional assist ventilation which supplies an assisting gas of an assisting gas pressure Pᵥₑₙₜ amplified proportionally to the respiratory effort pressure Pₘᵤₛ to the respiratory airway, to be attained more reliably. Moreover, a progress treatment in which the patient is separated from the gas-delivery mechanism can be attained preferably.

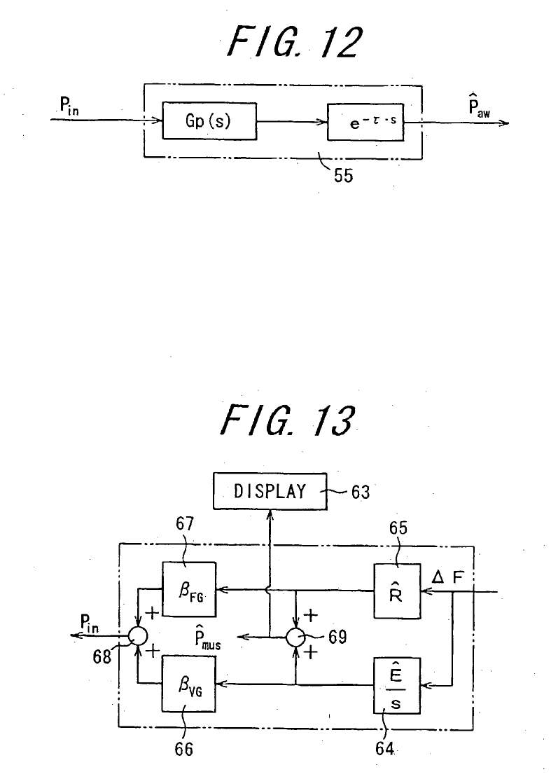

FIG. 12 is a block diagram showing a respiratory airway pressure calculation device 55. For example, the respiratory airway pressure calculation device 55 has a gas-delivery mechanism model obtained by simulating the gas-delivery mechanism 20. When a target pressure Pᵢₙ is given, the respiratory airway pressure calculation device 55 calculates a discharge pressure which the gas-delivery mechanism 20 will discharge, i.e. an assisting gas pressure Pᵥₑₙₜ. To be concrete, the transfer function Gp(s) set in the respiratory airway pressure calculation device 55 is set to a transfer function Gc(s)·e^{τ·s} obtained by simulating the transient characteristics of the gas-delivery mechanism 20.

When the transfer function Gp(s) of the respiratory airway pressure calculation device 55 is thus set, a calculated respiratory airway pressure \hat{P}_{aw} which varies with the lapse of time due to the transient characteristics of the gas-delivery mechanism 20 can be calculated.

The respiratory airway pressure calculation device 55 gives the calculated respirator pressure \hat{P}_{aw} to an observer 54. The respiratory airway pressure calculation device 55 can estimate the respiratory airway pressure \hat{P}_{aw} more accurately by taking into consideration a pressure resistance of the respiratory conduit, sampling time of the control apparatus and a pressure propagation delay.

FIG. 13 is a block diagram showing a control value calculation means 53. The control value calculation means 53 includes a volume calculation device 64, a flow-rate calculation device 65, a volume gain multiplier 66, a flow-rate gain multiplier 67, a first adder 68 and a second adder 69.

The volume calculation device 64 calculates a volume calculation value (Ê·ΔF/s) obtained by multiplying values, which are determined by integrating the flow-rate difference ΔF in order from the assisting gas supply starting time, by estimated elastance Ê. The volume gain multiplier 66 multiplies the predetermined volume amplification gain β_{VG} by the volume calculation value, and gives the resultant value to the first adder 68.

The flow-rate calculation device 65 calculates a flow-rate calculated value (ΔF·\hat{R}) obtained by multiplying a flow-rate difference ΔF, which is calculated by the difference calculation means 52, by an estimated respiratory resistance \hat{R}. The flow-rate gain multiplier 67 multiplies the predetermined flow-rate gain β_{FG} determined by the calculated value of flow-rate, and gives the resultant value to the first adder 68. The first adder 68 adds the calculated value given by the flow-rate gain multiplier 67 and that given by the volume multiplier 66 to each other, and the resultant value is given as a target pressure Pᵢₙ to the gas-delivery mechanism 20. This enables the volume amplification gain β_{VG} and flow-rate amplification gain β_{FG} to be set individually, and the convenience of the mechanical ventilator to be improved of its performances.

As shown in FIG. 13, the flow-rate calculation device 64 and volume calculation device 65 give the calculation results thereof respectively to the second adder 69. The second adder 69 adds the calculation result of the flow-rate calculation device 64 and that of the volume calculation device 65 to each other, and can determine the result as a respiratory effort pressure \hat{P}ₘᵤₛ. When the volume amplification gain β_{VG} and flow-rate amplification gain β_{FG} come to have an equal value β, a target pressure Pᵢₙ amplified by (1+β) times with respect to the calculated respiratory effort pressure \hat{P}mus can be achieved.

The control apparatus 21 may have a display unit 63. The display unit 63 obtains respiratory effort pressure \hat{P}ₘᵤₛ calculated by the second adder 69 of the spontaneous respiratory pressure estimation device 61, and can display the obtained respiratory effort pressure Pₘᵤₛ. Owing to what is thus displayed a doctor and so forth can ascertain the respiratory effort pressure Pₘᵤₛ which represents the strength of the respiration of the patient in a noninvasive manner.

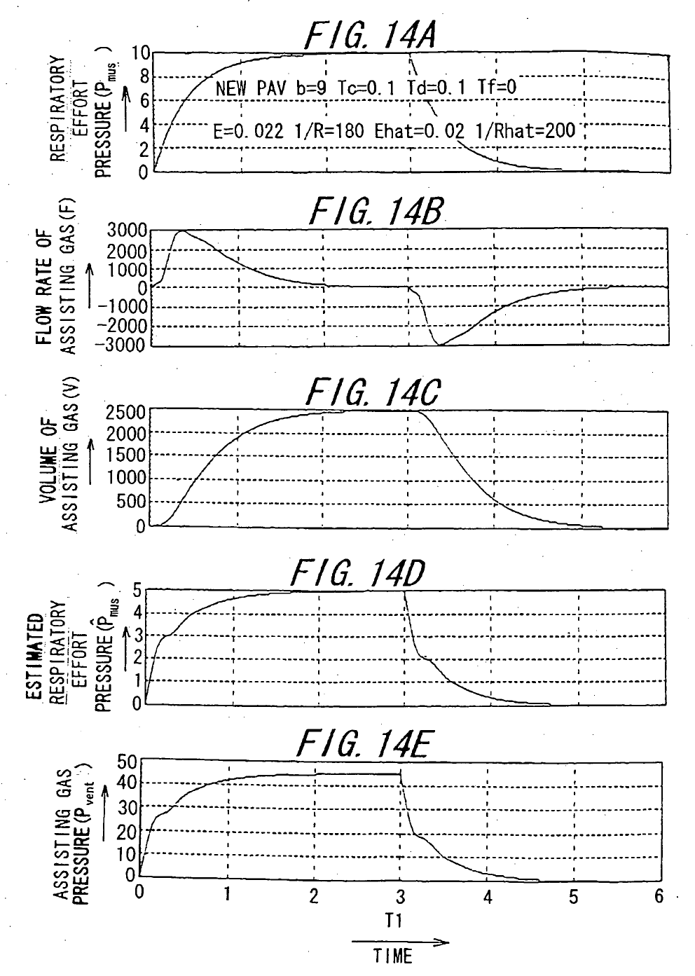

FIG. 14 shows the results of simulation of the overall system 14 in a case of embodiment of the invention. FIG. 14 shows the simulation results of the case where the control apparatus 21 has structures shown in FIG. 12 and FIG. 13. A case (Gc(s)·e^{-τ·s}≠1) where the estimated respiratory resistance \hat{R} and estimated elastance Ê have difference (\hat{R}≠R, Ê≠E) with respect to actual respiratory resistance R and actual elastance E with the transfer function of the gas-delivery mechanism 20 including a first-order delay factor and a dead time factor will be shown.

TABLE 1

| | | |
|---|---|---|
| Amplification factor | | 10 |
| Time constant of mechanical ventilator (sec) | Tc | 0.1 |
| Dead time of mechanical ventilator (sec) | τ | 0.1 |
| Actual respiratory resistance (cm H₂O/ml/sec)) | R | 0.022 |
| Actual elastance of lung (l/cm H₂O) | E | 1/180 |
| Estimated respiratory resistance (cm H₂O/ml/sec)) | \hat{R} | 0.02 |
| Estimated elastance (l/cm H₂O) | Ê | 1/200 |

Table 1 shows set values of parameters of the overall system of FIG. 14. In the simulation, are measured time variations in measured flow-rate F of the assisting gas, measured volume V of the assisting gas, estimated respiratory effort pressure \hat{P}ₘᵤₛ and assisting gas pressure Pᵥₑₙₜ in the case where a preset respiratory effort pressure Pₘᵤₛ is applied.

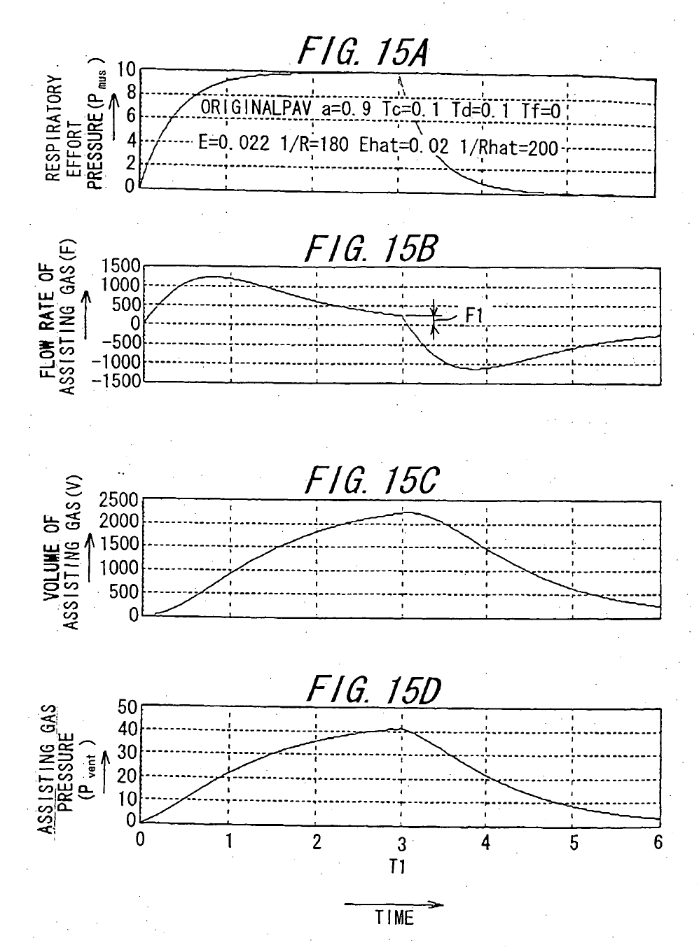

FIG. 15 show the results of simulation of the related art overall system 5. In FIG. 15, parameters are set correspondingly to those shown in FIG. 14. Namely, the flow-rate-assist gain K_{fa} is set to \hat{R}·α, volume gain K_{Va} is set to Ê·α, and amplification factor is set to 10. The other parameters are set identical with those in FIG. 14.

As shown in FIG. 15, the flow-rate F of the assisting gas does not quickly become zero as a combinational result of a un-negligible delay and positive feedback configuration in the related art overall system 5 at the inspiratory finishing time T1 when the inspiratory period of a patient terminates, and the assisting gas of a flow-rate shown by F1 in FIG. 15(4) is necessarily supplied to the respiratory airway of the patient. Namely, even after the condition of the patient is starting exhalation breath after ending of inspiration, the assisting gas is supplied to the patient, and the patient is put in a so-called asynchronous condition. When the patient is put in an asynchronous condition, a burden on the patient becomes large.

As shown in FIGS. 15(1) and 15(4), as a combinational result of a un-negligible delay and positive feedback configuration in the overall system 5, the waveform of the assisting gas pressure Pᵥₑₙₜ does not become a waveform proportional to that of the respiratory effort pressure Pₘᵤₛ. This also causes the burden on the patient to become large.

On the other hand, in the overall system 14 according to the invention, the assisting gas pressure Pᵥₑₙₜ is set in accordance with the transient characteristics of the gas-delivery mechanism 20, so that the measured flow-rate F of the assisting gas becomes zero at the inspiratory finishing time T1 as shown in FIG. 14(2). In the overall system 14 according to the invention, it is possible to set or regulate the overall system so that the possibility of occurrence of the asynchronous condition is reduced to a low level, and the supplying of the assisting gas can be done only in the inspiratory period corresponding to the respiratory effort pressure Pₘᵤₛ of the patient.

As shown in FIG. 14(1) and FIG. 14(5), the assisting gas pressure Pᵥₑₙₜ proportional to the respiratory effort pressure Pₘᵤₛ can be given. In the overall system according to the invention, the asynchronous condition does not occur even when the amplification gain β is increased. Moreover, when the estimated respiratory resistance \hat{R} and estimated elastance Ê have errors with respect to the actual estimated respiratory resistance R and elastance E, an asynchronous condition does not occur. When the occurrence of the asynchronous condition is thus prevented, the artificial respiration can be carried out with a burden on the patient further reduced.

The reason why the assisting gas pressure Pᵥₑₙₜ proportional to the respiratory effort pressure Pₘᵤₛ can be given with the asynchronous condition thus eliminated resides in that the control apparatus 21 is provided therein with a model made by estimating a pressure transmission lag due to the gas-delivery mechanism 20 and an air circuit, on the basis of which model the flow-rate estimated value \hat{F} is determined.

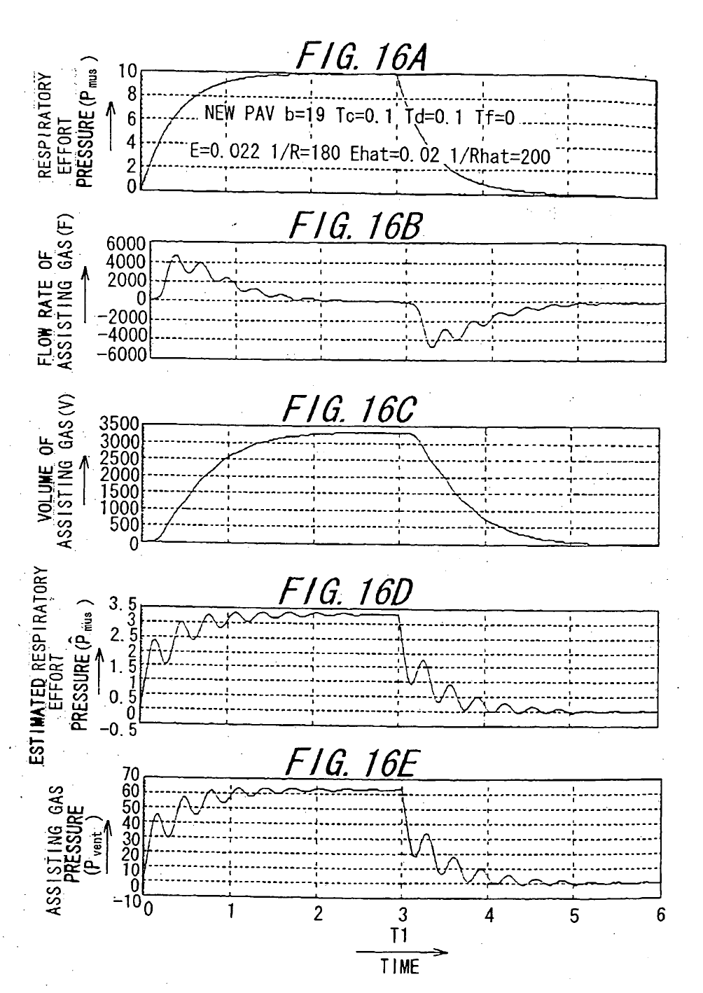

FIG. 16 shows results of simulation of a case where, out of the parameters shown in Table 1, the amplification gain β is changed to 19. When the amplification gain β is increased extremely as shown in FIG. 16, the flow-rate F of the assisting gas becomes vibratory as shown in FIG. 16(2) in the overall system 14 in the mode of embodiment. It is not preferable that the assisting gas pressure Pᵥₑₙₜ becomes vibratory in this manner.

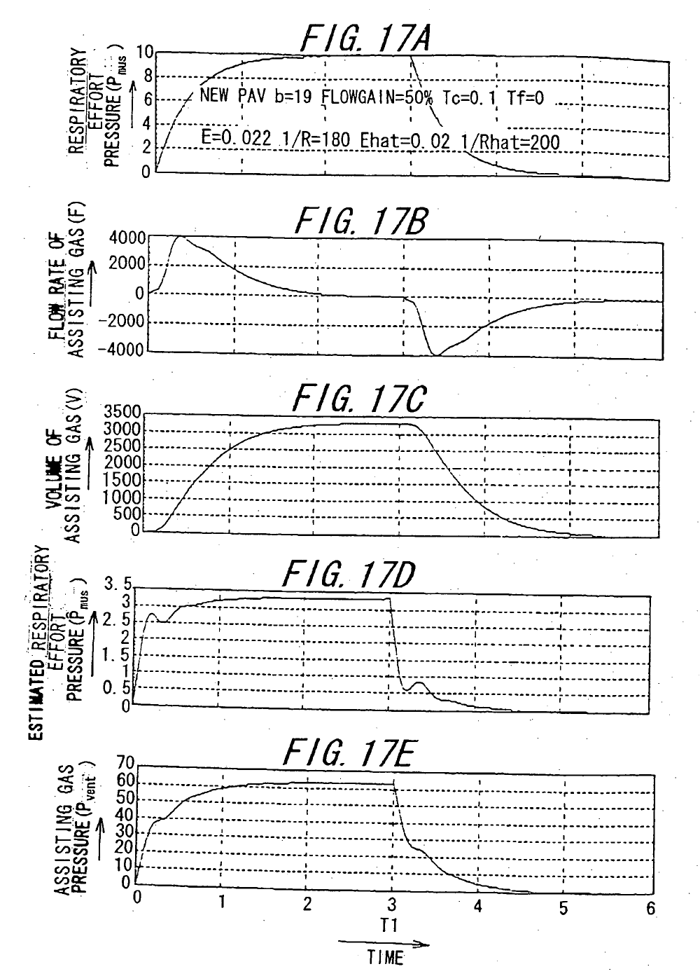

FIG. 17 show the results of the simulation of a case where the flow-rate calculation value of the flow-rate amplification gain β_{FG} in the condition shown in FIG. 16 is reduced to 50% by the flow-rate gain multiplier 67. When the flow-rate F of the assisting gas is vibratory, the flow-rate amplification gain β_{FG} is reduced, and the resultant value is given to the first adder 68, so that the flow-rate of the assisting gas can be prevented from becoming vibratory as shown in FIG. 17. This enables the amplification factor to be increased without causing the flow-rate of the assisting gas to become vibratory.

The flow-rate amplification gain β_{FG} corresponds to the proportional gain in the PI control operation. Therefore, when the flow-rate amplification gain β_{FG} is regulated, the quick-responsibility with respect to the respiratory effort pressure Pₘᵤₛ can be improved. The volume amplification gain β_{VG} corresponds to the integration gain in the PI control operation. Therefore, when the volume amplification gain β_{VG} is regulated, the steady gain of the target pressure Pᵢₙ can be regulated.

When the volume amplification gain β_{VG} and flow-rate gain β_{FG} are thus regulated, the target pressure Pᵢₙ can be set with the control characteristics, such as the adaptability and attenuation coefficients including the steady-state gain improved. Since the stability of the overall system 14 according to the invention is improved as mentioned above, the degree of freedom of selecting parameters is large, so that runaway is rarely occurs even when the amplification gain β is increased, and even when the volume amplification gain β_{VG} and flow-rate amplification gain β_{FG} are changed, the regulation operation being thereby able to be carried out suitably.

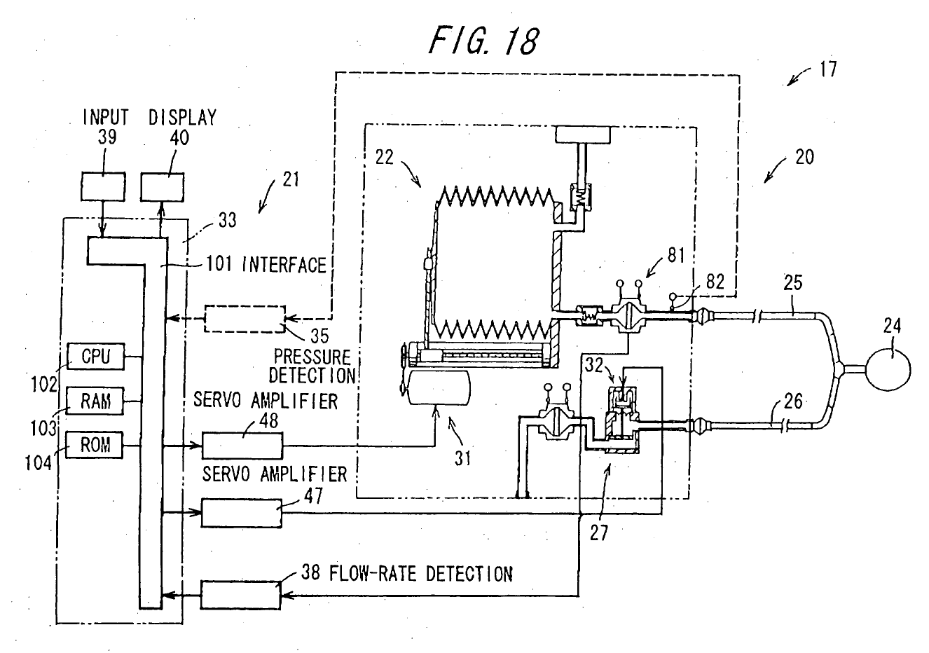

FIG. 18 is a block diagram showing an example of a mechanical ventilator 17. A control apparatus 21 includes a main body 33 of a computer control apparatus, a flow-rate measuring means 50, an input unit 39, a display 40 and servo amplifiers 47, 48 with amplifier circuits. The control apparatus 21 may include a respiratory airway pressure measuring means 61.

The flow-rate measuring means 50 is adapted to convert a flow-rate of a gas flowing in an inspiratory conduit 25 of a gas-delivery mechanism 20 into an electric signal, which is given to the control apparatus main body 33. To the input unit 39 are inputted receive estimated respiratory resistance \hat{R}, estimated elastance Ê, amplification gain β, time constant Tc, dead time τ and the like of the gas-delivery mechanism 20 by a person who controls the gas-delivery mechanism 20, such as a doctor and a therapist. The input unit 39 is adapted to give a signal representative of inputted information to the control apparatus main body 33.

The display 40 is a man-machine interactive device for informing the respiratory airway pressure of a patient. The display 40 is adapted to show a waveform indicating the variation with the lapse of time of the respiratory effort pressure Pₘᵤₛ of the patient on a picture frame on the basis of a display instruction signal received from the control apparatus main body 33.

The amplifier circuit 48 is adapted to give a signal representative of a target pressure Pᵢₙ calculated by the control apparatus main body 33 to an actuator 31 for a pump. The pump actuator 31 is adapted to control the pump on the basis of a signal representative of the target pressure Pᵢₙ, and feedback control the discharge pressure of the gas-delivery mechanism 20.

The control apparatus main body 33 includes an interface 101, a calculation portion 102, a temporary storage 103 and a storage 104. The interface 101 is adapted to receive a signal from a flow-rate measuring means 50 connected thereto, and give the signal to the calculation portion 102. The storage 104 is adapted to store a program to be executed by the control apparatus main body 33 and the calculation portion 102 reads out a program stored in the storage 104 and execute the same, the flow-rate estimation means 51, difference calculation means 52 and control value calculation means 53 being thereby rendered executable. This enables the control apparatus main body 33 to control the gas-delivery mechanism 20. The storage 104 may be a recording medium readable by a computer, such as a compact disk.

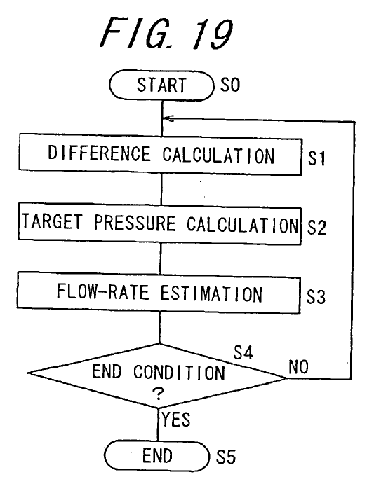

FIG. 19 is a flow chart showing operations of a control apparatus main body 33. The control apparatus main body 33 first receives in a step s0 parameters, such as estimated respiratory resistance \hat{R}, estimated elastance Ê, transfer function of the gas-delivery mechanism 20, flow-rate gain K_{FG} and volume gain K_{VG}. When it becomes possible to calculate a target pressure Pᵢₙ and estimated flow-rate \hat{F} and prepare the calculation thereof, the process advances to a step s1.

In the step s1, the control apparatus main body 33 carries out operations of the difference calculation means 52, and calculates a difference ΔF between the estimated flow-rate \hat{F} determined on the basis of the previously calculated target pressure Pᵢₙ and the flow-rate F given from the flow-rate measuring means 50. When the calculation of the flow-rate difference ΔF finishes, the process advances to a step s2.

In the step s2, the control apparatus main body 33 carries out the operations of the control value calculation means 53, and calculates the target pressure Pᵢₙ on the basis of the flow-rate difference ΔF. When the calculation of the target pressure Pᵢₙ finishes, a signal representative of the target pressure Pᵢₙ is given to the gas-delivery mechanism 20, and the process advances to the step s3.

In the step s3, the control apparatus main body 33 carries out the operations of the flow-rate estimation means 51, and calculates estimated flow-rate \hat{F} which will be supplied to the patient when a signal representative of the target pressure Pᵢₙ is input to the gas-delivery mechanism 20, and the process advances to a step s4.

In the step s4, the control apparatus main body 33 judges whether predetermined finishing condition is satisfied or not. For example, when a finishing instruction is not given from the input unit, a judgment that the controlling of the gas-delivery mechanism 20 is continued is given, and the process returns to the step s1. In the step s1, the flow-rate difference ΔF is calculated again by using the estimated flow-rate \hat{F} calculated in the step s3 and the flow-rate F given from the flow-rate measuring means 50. When the control apparatus main body 33 judges in the step s4 that the predetermined finishing condition is satisfied, the process advances to the step s5 to finish the control operations.

The gas-delivery mechanism 20 is capable of controlling the pressure of the discharged assisting gas by the control apparatus 21, and not specially limited as long as the gas-delivery mechanism 20 is provided with the inspiratory conduit 25 for introducing the assisting gas into a respiratory airway 15 of a patient. For example, the gas-delivery mechanism 20 may be a mechanical ventilator having bellows type pump as shown in FIG. 18. Also, a mechanical ventilator adapted to supply an assisting gas via a pipe is available.

The transient characteristics of the gas-delivery mechanism 20 do not vary greatly as compared with the condition of the patient, so that the characteristics can be determined in advance. For example, the transfer function of the gas-delivery mechanism 20 is determined, and factors of the transfer function are set in the respiratory pressure calculation device 55. Similarly, the measuring lag of the flow-rate measuring means is also determined in advance, and the result is given to the control apparatus 21.

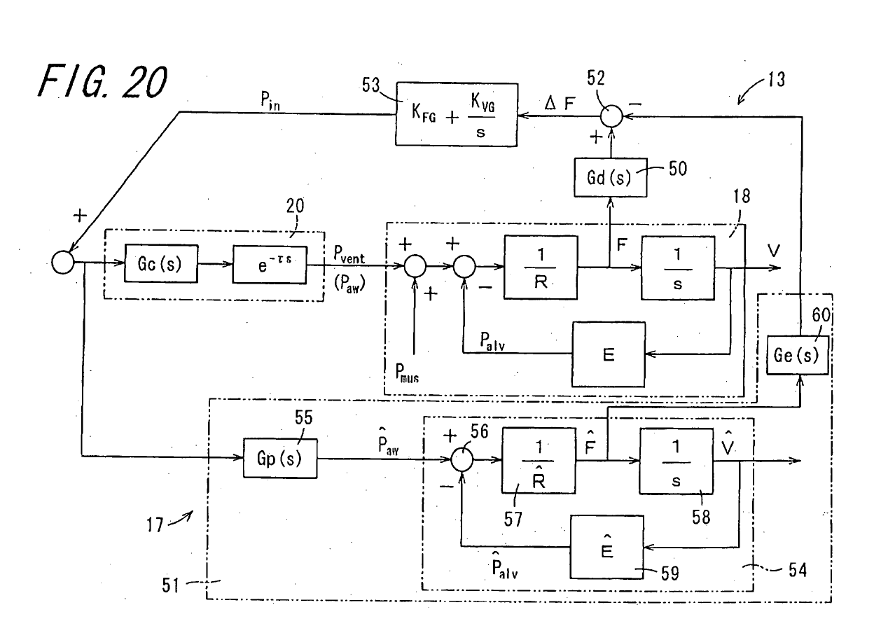

FIG. 20 is a block diagram showing an overall system 13 in a case of another embodiment of the invention. The overall system 13 shown in FIG. 20 has a structure identical with that of the overall system 14 shown in FIG. 3 except that a part of the structure of the flow-rate estimation means 51 is different. Therefore, a description of the identical structure will be omitted, and reference numerals corresponding to those for the overall system 14 of FIG. 2 will be added.

A flow-rate estimation means 51 further has a measuring delay calculation device 60. The measuring delay calculation device 60 has a measuring means model obtained by simulating the flow-rate measuring means 50. When an estimated flow-rate \hat{F} is given from an observer 54 to the measuring lag calculation device 60, an estimated flow-rate \hat{F} based on the measuring delay of the flow-rate measuring means 50 is calculated, and the result of this calculation is given to the difference calculation means 52. The difference calculation means 52 subtracts the flow-rate measured by the flow-rate measuring means 50 from estimated flow-rate \hat{F} calculated by the measuring lag calculation device 60, and a flow-rate difference ΔF is calculated. This enables the flow-rate \hat{F} of the assisting gas to be supplied to the respiratory airway of the patient to be calculated with a further high accuracy on the basis of the time characteristics from the time at which a flow-rate F of an assisting gas supplied to the respiratory airway of a patient is measured to the time at which the measuring means 50 outputs the result of the measuring. Therefore, the occurrence of non-synchronous condition can be prevented more reliably.

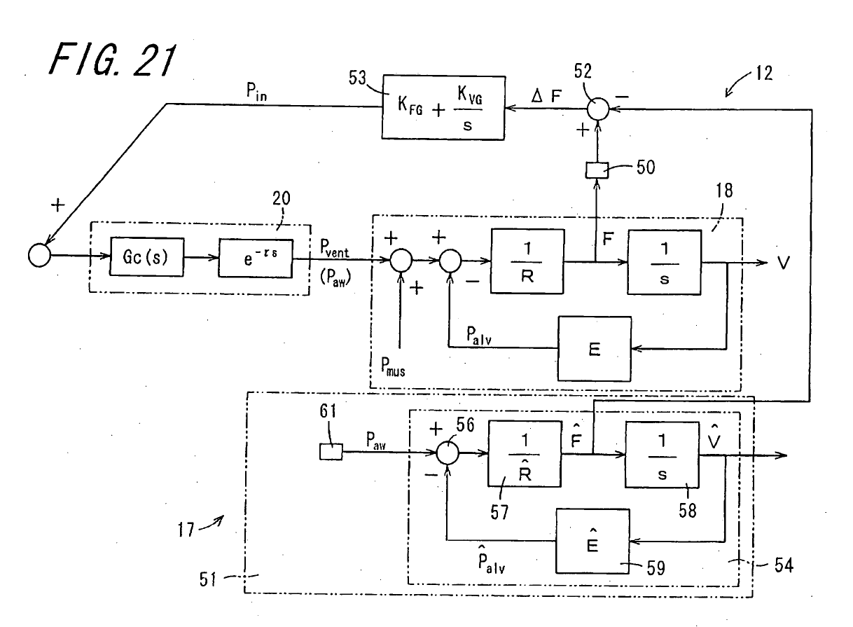

FIG. 21 is a block diagram showing an overall system 12 of still another mode of embodiment of the invention. The overall system 12 shown in FIG. 21 has a structure identical with that of the overall system 14 shown in FIG. 3 except that a part of the structure of the flow-rate estimation means 51 is different. Therefore, a description of the same structure will be omitted, and reference numerals corresponding to those for the overall system 14 of FIG. 3 will be added.

Instead of the flow-rate estimation means 51, a pressure measuring means 61 may be provided. The pressure measuring means 61 is adapted to detect a pressure P_{aw} in the respiratory airway of a patient. The pressure measuring means 61 gives the measured respiratory airway pressure P_{aw} to an observer 54. Even such a structure can attain the above-mentioned effect. When the respiratory airway pressure P_{aw} is measured, the respirators airway pressure P_{aw} can be obtained without taking the influence of the delay of the gas-delivery mechanism 20 into consideration, and an assisting gas can be given to the respiratory airway under an assisting gas pressure Pᵥₑₙₜ accurately corresponding to a respiratory effort pressure Pₘᵤₛ.

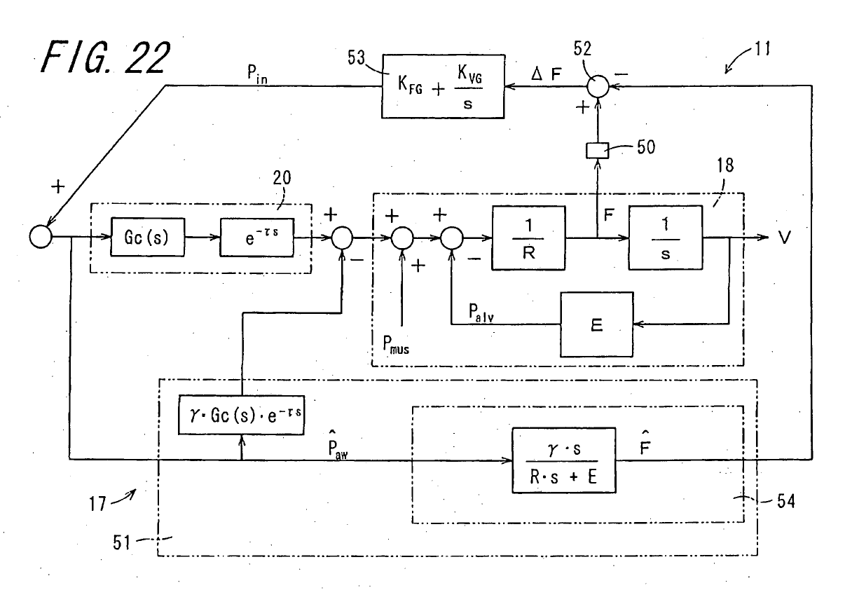

FIG. 22 is a block diagram showing an overall system 11 of a further mode of embodiment of the invention. The overall system 11 shown in FIG. 22 is an equivalently converted model concerning a case where the transfer function Gp(s) set in the respiratory airway pressure calculation device 55 in the overall system 14 shown in FIG. 2 is expressed by the following equation (15).

$$Gp ( s ) = γ \cdot \frac{\hat{R} \cdot s + \hat{E}}{R \cdot s + E} [ a + Gc ( s ) \cdot {ⅇ}^{- τ s} ] \qquad ( 15 )$$

In this equation, γ is a coefficient set in 0<γ<1. This transfer function Gp(s) indicates a case where the actual respiratory resistance R and actual elastance E are already known. When the actual respiratory resistance R and actual pulmonary elastance E are not known, the following equation is substituted for the equation (15).
Gp(s)=γ−└1+Gc(s)·e^{-τ·s}┘ (16)

When the transfer function of the respiratory airway pressure calculation device 55 is thus set to the level equal to that in the overall system 11 shown in FIG. 22, a delay due to the dead time factor of the gas-delivery mechanism 20 can be made up for by a factor of γ·Gc(s)·e^{-τ·s}, and the delay due to the dead time of the gas-delivery mechanism can be suitably compensated.

The respiratory airway pressure calculation device 55 may be formed such that the calculation device can thus estimate the respiratory airways pressure P_{aw} on the basis of the target pressure Pᵢₙ. Therefore, the transfer function Gp(s) of the respiratory airway pressure calculation device 55 may be set to a level other than that of a transfer function Gc(s) which is obtained by simulating the characteristics of the gas-delivery mechanism 20.

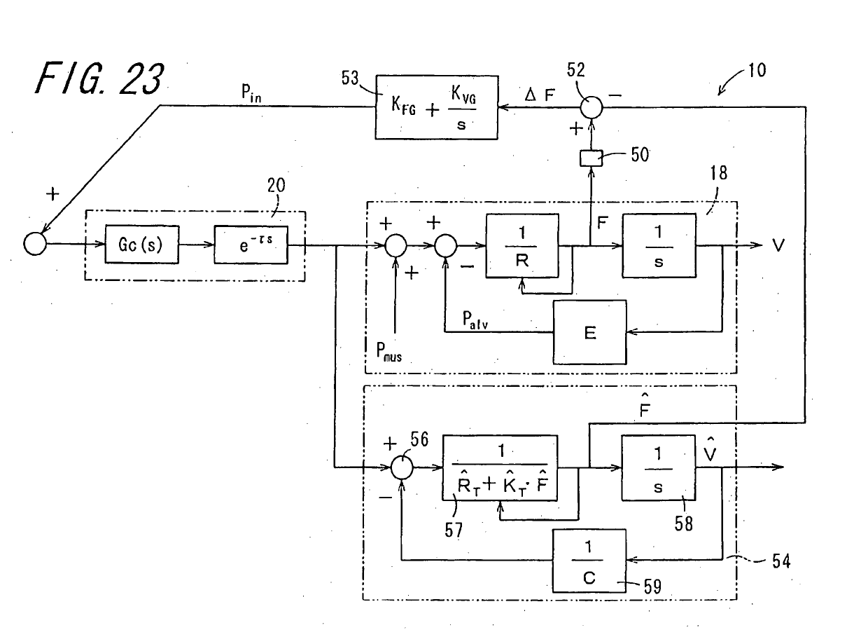

FIG. 23 is a block diagram of an overall system 10 in another mode of embodiment of the invention. The overall system 10 shown in FIG. 23 has a structure identical with that of the overall system 14 shown in FIG. 3 except that the setting of an estimated respiratory resistance \hat{R} and estimated elastance Ê which are set in the flow-rate estimation means 51 is different. Therefore, a description of the identical structure will be omitted, and reference numerals corresponding to those used for the overall structure 14 of FIG. 3 will be added.

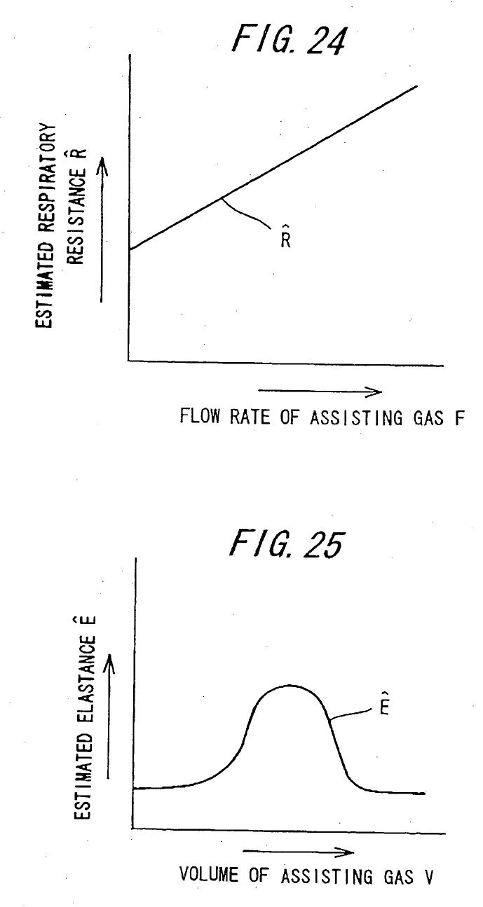

FIG. 24 is a graph for describing the respiratory resistance R. When a flow of an assisting gas in the respiratory airway becomes a laminar flow, the velocity of flow thereof varies linearly in proportion to the respiratory airway pressure P_{aw}. However, the respiratory airway branches repeatedly in practice, and the diameter thereof is not uniform, so that the flow of the assisting gas becomes turbulence. Therefore, the estimated respiratory resistance \hat{R} is set taking the turbulence resistance into consideration.

The estimated respiratory resistance \hat{R} set in the flow-rate estimation means 51 is a value obtained by summing up a first resistance factor \hat{R}_{T} set constantly irrespectively of a flow rate of the assisting gas and a second resistance factor \hat{K}_{T} depending on the flow rate \hat{F} of the assisting gas calculated by the estimated flow-rate calculation device. The first resistance factor \hat{R}_{T} and second resistance factor \hat{K}_{T} are set to levels according to the respiratory resistance of a patient. The resistance of the respiratory system as a whole including not only the lung but also the thorax may be set as the estimated respiratory resistance \hat{R}. The respiratory resistance R may be drawn toward an estimated respiratory resistance \hat{R} represented by other approximate expression.

FIG. 25 is a graph for describing the compliance of the lung. The estimated elastance Ê set in the flow-rate estimation means 51 is a value based on the assisting gas volume \hat{V} calculated by the above-mentioned assisting gas volume calculation device, and this value becomes an inverse of the compliance C of the lung. The compliance C increases non-linearly with an increase of the volume V of the assisting gas during the inspiratory period of the patient, and has saturation characteristics and hysteresis characteristics.

The alveoli pressure calculation device 59 obtains in advance information representative of the relation between the compliance C and the volume of the assisting gas. Owing to the obtainment of such information, the alveoli pressure Pₐₗᵥ can be calculated accurately even though a case where the compliance is non-linear is taken into consideration.

When the observer has a model of respiratory system which thus becomes more non-linear, the flow rate \hat{F} can be estimated more accurately. This enables the respiratory effort pressure Pₘᵤₛ to be estimated with a high accuracy, and the assisting gas pressure Pᵥₑₙₜ to be determined in accordance with the estimated respiratory effort pressure Pₘᵤₛ.

The above-described modes of embodiment of the invention are examples thereof, and the construction thereof can be modified within the scope of the invention. For example, the above-mentioned block diagrams are merely examples of the invention, and may be equivalently chanced when the same effect can be obtained. When a computer to which the measured flow rate F is given calculates a target pressure Pᵢₙ, each unit may be formed in practice by software.

The estimated respiratory resistance \hat{R} and estimated elastance Ê may be set suitably by a doctor, and the respiratory resistance R and elastance E which are measured in advance with measuring instruments may also be used. Moreover, the respiratory resistance \hat{R} and elastance Ê determined by the estimating method disclosed in JP-A 11-502755 may also be used.

The invention may be embodied in other specific forms without departing from the spirit or essential characteristics thereof. The embodiments are therefore to be considered in all respects as illustrative and not restrictive, the scope of the invention being indicated by the appended claims rather than by the foregoing description and all changes which come within the meaning and the range of equivalency of the claims are therefore intended to be embraced therein.

1. A method for controlling a gas-delivery mechanism of a mechanical ventilator which supplies a bas containing oxygen having an assisting gas pressure Pᵥₑₙₜ corresponding to patient's respiratory effort pressure Pₘᵤₛ, the method comprising:
a flow-rate measuring step of measuring a flow rate F of an assisting gas supplied to a patient's respiratory airway;
a flow-rate estimation step of estimating a flow rate \hat{F} of an assisting gas to be supplied to the patient's respiratory airway when the assisting gas having an assisting gas pressure Pᵥₑₙₜ is supplied to the patient's respiratory airway, with the aid of flow-rate estimation means in which a patient's respiratory system is modeled; a difference calculation step of calculating a flow-rate difference ΔF between the measured flow rate F and the estimated flow rate \hat{F}; and
a control value calculation step of calculating the target pressure Pᵢₙ, based on the flow-rate difference ΔF and providing a signal representing the target pressure Pᵢₙ, to the gas-delivery mechanism.

2. The method of claim 1, wherein the assisting gas flow rate \hat{F} to a patient is estimated based on a series of time-courses that the target pressure signal Pᵢₙ is calculated and thereafter transmitted to the gas-delivery mechanism, and then this gas-delivery mechanism consequently delivers the assisting gas having an assisting gas pressure Pᵥₑₙₜ.

3. A control apparatus for controlling a gas-delivery mechanism of a mechanical ventilator which supplies a gas containing oxygen having an assisting gas pressure Pᵥₑₙₜ corresponding to patient's respiratory effort pressure Pₘᵤₛ, the method comprising:
flow-rate measuring means for measuring a flow rate F of an assisting gas to be supplied to a patient's respiratory airway;
flow-rate estimation means for estimating a flow-rate \hat{F} of an assisting gas to be supplied to the patient's respiratory airway when the assisting gas having an assisting gas pressure Pᵥₑₙₜ is supplied to the patient's respiratory airway, in the flow rate estimation means a patient's respiratory system being modeled;
difference calculation means for calculating a flow-rate difference ΔF between the measured flow rate F and the estimated flow rate \hat{F}; and
control value calculation means for calculating the target pressure Pᵢₙ based on the flow-rate difference ΔF and providing a signal representing the target pressure Pᵢₙ to the gas-delivery mechanism.

4. The control apparatus of claim 3, wherein the float-rate estimation means has a gas-delivery mechanism model obtained by modeling the gas-delivery mechanism, and the assisting gas flow rate \hat{F} to a patient is estimated based on a series of time-courses that the target pressure signal Pᵢₙ is calculated and thereafter transmitted to the gas-delivery mechanism, and then this gas-delivery mechanism consequently delivers the assisting gas having an assisting gas pressure Pᵥₑₙₜ.

5. The control apparatus of claim 3, wherein the flow-rate estimation means has measuring means model obtained by modeling a flow-rate measuring means, and a flow rate to be delivered to a patient's respiratory airway \hat{F} is estimated based on a series of time-courses that the assisting gas having been delivered to a patient's respiratory airway is measured, and then a measuring result of the measuring means is outputted from the measuring means.

6. The control apparatus of 3, wherein the control value calculation means determines a first calculation value (K_{FG}·ΔF) which is a product of a predetermined flow-rate gain K_{FG} and the flow-rate difference ΔF, and a second calculation value (K_{FG}·ΔF/s) whish is a product of a predetermined volume gain K_{VG}, and an integral of ΔF, and
then adds the first calculation value (K_{FG}·ΔF) and the second calculation value (K_{FG}·ΔF/s) to calculate the target pressure Pᵢₙ.

7. The control apparatus of claim 3, wherein the flow-rate estimation means further comprises a respiratory airway pressure calculation device for calculating patient's respiratory airway pressure \hat{P}_{aw}, and
the respiratory system model includes:
a subtracter for subtracting an alveolar pressure \hat{P}ₐₕ induced by elastic lung-recoil pressure from the respiratory airway pressure \hat{P}_{aw} calculated by the respiratory airway pressure calculation device when a patient's respiratory effort pressure Pₘᵤₛ does not exist;
an estimated flow-rate calculation device for estimating a flow rate \hat{F} of the assisting gas to be delivered to the patient's respiratory airway by dividing the subtracted value obtained by the subtracter by an estimated patient's respiratory resistance \hat{R};
an assisting gas volume calculation device for calculating a volume \hat{V} of the assisting gas to be delivered to the patient's respiratory airway by integrating successively the flow rate of the assisting gas \hat{F} from starting time of delivering the assisting gas: and
an alveolar pressure calculation device for calculating alveolar pressure Pₐₗᵥ by multiplying the calculated volume \hat{V} of the assisting gas by an estimated respiratory elastance Ê to supply the calculated alveolar pressure \hat{P}ₐₗᵥ to the subtracter.

8. The control apparatus of claim 7, wherein the estimated patient's respiratory resistance \hat{R} is a sum of a first resistance coefficient \hat{R}_{T} which is constant regardless of a flow rate of the assisting gas, and a second resistance coefficient \hat{K}_{T} which is based on the flow rate \hat{F} of the assisting gas calculated by the estimated flow-rate calculation device, and
the estimated respiratory elastance Ê is a value based on the volume \hat{V} of the assisting gas calculated by the assisting gas volume calculation device.

9. The control apparatus of claim 7, further comprising modifying means for modifying at least one of the estimated patient's respiratory resistance \hat{R} and the estimated respiratory elastance Ê, based on either the flow rate \hat{F} of the assisting gas having been delivered to the patient's respiratory airway or an input value inputted from an outside.

10. The control apparatus of claim 3, further comprising pressure measuring means for measuring the assisting gas pressure Pᵥₑₙₜ,
wherein the flow-rate estimation means estimates a flow rate \hat{F} of the assisting gas to be delivered to the patient's respiratory airway based on the assisting gas pressure Pᵥₑₙₜ, measured by the pressure measuring means.

11. A patient's respiratory effort pressure estimation apparatus for estimating a patient's respiratory effort pressure Pₘᵤₛ when an assisting gas containing oxygen is delivered to a patient's respiratory airway with a predetermined assisting gas pressure Pᵥₑₙₜ, comprising:
flow-rate measuring means for measuring a flow rate \hat{F} of the assisting gas having been delivered to the patient's respiratory airway:
flow-rate estimation means having a respiratory system model obtained by modeling a patient's respiratory system, for estimating a flour rate \hat{F} of the assisting gas to be delivered to the patient's respiratory airway when the assisting gas is delivered thereto with the assisting gas pressure Pᵥₑₙₜ;
difference calculation means for calculating a flow-rate difference ΔF between the measured flow rate F and the estimated flow rate \hat{F}; and
respiratory effort pressure estimation means for estimating the patient's respiratory effort pressure Pₘᵤₛ based on the flow-rate difference ΔF.

12. The control apparatus of claim 4, wherein the flow-rate estimation means has measuring means model obtained by modeling a flow-rate measuring means, and a flow rate to be delivered to a patient's respiratory airway \hat{F} is estimated based on a series of time-courses that the assisting gas having been delivered to a patient's respiratory airway is measured, and then a measuring result of the measuring means is outputted from the measuring means.

13. The control apparatus of claim 4, wherein the control value calculation means determines a first calculation value (K_{FG}·ΔF) which is a product of a predetermined flow-rate gain K_{FG} and the flow-rate difference ΔF, and a second calculation value (K_{FG}·ΔF/s) whish is a product of a predetermined volume gain K_{VG}, and an integral of ΔF, and
then adds the first calculation value (K_{FG}·ΔF) and the second calculation value (K_{FG}·ΔF/s) to calculate the target pressure Pᵢₙ.

14. The control apparatus of claim 5, wherein the control value calculation means determines a first calculation value (K_{FG}·ΔF) which is a product of a predetermined flow-rate gain K_{FG} and the flow-rate difference ΔF, and a second calculation value (K_{FG}·ΔF/s) whish is a product of a predetermined volume gain K_{VG}, and an integral of ΔF, and
then adds the first calculation value (K_{FG}·ΔF) and the second calculation value (K_{FG}·ΔF/s) to calculate the target pressure Pᵢₙ.

15. The control apparatus of claim 3, wherein the flow-rate estimation means further comprises a respiratory airway pressure calculation device for calculating patient's respiratory airway pressure \hat{P}_{aw}, and
the respiratory system model includes:
a subtracter for subtracting an alveolar pressure \hat{P}ₐₕ induced by elastic lung-recoil pressure from the respiratory airway pressure \hat{P}_{aw} calculated by the respiratory airway pressure calculation device when a patient's respiratory effort pressure Pₘᵤₛ does not exist;
an estimated flow-rate calculation device for estimating a flow rate \hat{F} of the assisting gas to be delivered to the patient's respiratory airway by dividing the subtracted value obtained by the subtracter by an estimated patient's respiratory resistance \hat{R};
an assisting gas volume calculation device for calculating a volume \hat{V} of the assisting gas to be delivered to the patient's respiratory airway by integrating successively the flow rate of the assisting gas \hat{F} from starting time of delivering the assisting gas: and
an alveolar pressure calculation device for calculating alveolar pressure Pₐₗᵥ by multiplying the calculated volume \hat{V} of the assisting gas by an estimated respiratory elastance Ê to supply the calculated alveolar pressure \hat{P}ₐₗᵥ to the subtracter.

16. The control apparatus of claim 5, wherein the flow-rate estimation means further comprises a respiratory airway pressure calculation device for calculating patient's respiratory airway pressure \hat{P}_{aw}, and
the respiratory system model includes:
a subtracter for subtracting an alveolar pressure \hat{P}ₐₕ induced by elastic lung-recoil pressure from the respiratory airway pressure \hat{P}_{aw} calculated by the respiratory airway pressure calculation device when a patient's respiratory effort pressure Pₘᵤₛ does not exist;
an estimated flow-rate calculation device for estimating a flow rate \hat{F} of the assisting gas to be delivered to the patient's respiratory airway by dividing the subtracted value obtained by the subtracter by an estimated patient's respiratory resistance \hat{R};
an assisting gas volume calculation device for calculating a volume \hat{V} of the assisting gas to be delivered to the patient's respiratory airway by integrating successively the flow rate of the assisting gas \hat{F} from starting time of delivering the assisting gas: and
an alveolar pressure calculation device for calculating alveolar pressure Pₐₗᵥ by multiplying the calculated volume \hat{V} of the assisting gas by an estimated respiratory elastance Ê to supply the calculated alveolar pressure \hat{P}ₐₗᵥ to the subtracter.

17. The control apparatus of claim 6, wherein the flow-rate estimation means further comprises a respiratory airway pressure calculation device for calculating patient's respiratory airway pressure \hat{P}_{aw}, and the respiratory system model includes:
a subtracter for subtracting an alveolar pressure \hat{P}ₐₕ induced by elastic lung-recoil pressure from the respiratory airway pressure \hat{P}_{aw} calculated by the respiratory airway pressure calculation device when a patient's respiratory effort pressure Pₘᵤₛ does not exist;
an estimated flow-rate calculation device for estimating a flow rate \hat{F} of the assisting gas to be delivered to the patient's respiratory airway by dividing the subtracted value obtained by the subtracter by an estimated patient's respiratory resistance \hat{R};
an assisting gas volume calculation device for calculating a volume \hat{V} of the assisting gas to be delivered to the patient's respiratory airway by integrating successively the flow rate of the assisting gas \hat{F} from starting time of delivering the assisting gas: and
an alveolar pressure calculation device for calculating alveolar pressure Pₐₗᵥ by multiplying the calculated volume \hat{V} of the assisting gas by an estimated respiratory elastance Ê to supply the calculated alveolar pressure \hat{P}ₐₗᵥ to the subtracter.

18. The control apparatus of claim 8, further comprising modifying means for modifying at least one of the estimated patient's respiratory resistance \hat{R} and the estimated respiratory elastance Ê, based on either the flow rate \hat{F} of the assisting gas having been delivered to the patient's respiratory airway or an input value inputted from an outside.
